(19) United States
(12) Patent Application Publication
(10) Pub. No.: US 2026/0240460 A1
(43) Pub. Date: Aug. 20, 2026

(54) SPATIALLY RESOLVED DIFFUSE REFLECTANCE FOR BILIRUBIN MEASUREMENT

(71) Applicant: Temple University - Of The Commonwealth System of Higher Education, Philadelphia, PA (US)

(72) Inventors: Chetan A. Patil, Haddonfield, NJ (US); Alex Dumont, Granby, CT (US); Brandon Harrison, Philadelphia, PA (US); Shahriar Arefin, Philadelphia, PA (US)

(21) Appl. No.: 19/165,235

(22) PCT Filed: Apr. 11, 2024
(86) PCT No.: PCT/US2024/023977

§ 371 Date: Sep. 15, 2025

Related U.S. Application Data
(60) Provisional application No. 63/495,313, filed on Apr. 11, 2023.

(51) Int. Cl. A61B 5/1455; A61B 5/00; A61B 5/145

(52) U.S. Cl. CPC A61B 5/1455; A61B 5/0082; A61B 5/14546; A61B 5/6898; A61B 5/7225; A61B 2560/0223

(57) ABSTRACT

A 2D image sensor system for optical measurements of physiological parameters through a patient's skin using spatially resolved diffuse reflectance. The system includes a 2D image sensor adapted to be placed near/against the skin with at least three wavelength-dependent spectral channels and a field of view (FOV) with multiple spatial regions of interest (ROI). A source directs light onto the skin at a distance offset from the FOV. A detector receives light reflected from the skin and collects a 2D map of the entire reflectance spatial decay profile characteristic of the skin in different detector channels. A selector identifies multiple ROI that can be uniquely positioned in each spectral channel and/or partially overlapping in the 2D reflectance spatial decay map as the elements of a multivariate regression model to approximate a physiological parameter. The increased dimensionality of the system outputs improves measurement accuracy and reduces bias in confounding optical factors.

### CROSS-REFERENCE TO RELATED APPLICATIONS

This application claims priority to U.S. provisional Application No. 63/495,313 filed on Apr. 11, 2023, incorporated herein by reference in its entirety.

### TECHNICAL FIELD

The present disclosure relates generally to optical diagnostic devices and, more particularly, to systems for transcutaneous bilirubin measurements that use spatially resolved diffuse reflectance.

### BACKGROUND OF THE DISCLOSURE

Optical diagnostic devices such as transcutaneous bilirubin (TcB) devices have become standard throughout medicine. TcB devices estimate bilirubin levels in the blood. Both techniques are real-time, noninvasive surrogates for gold-standard blood tests, and in practice help physicians make timely decisions for clinical management.

Recently, there has been gathering attention concerning the accuracy of these transcutaneous optical devices in dark-skinned individuals. Studies have shown that TcB devices suffer from systematic overestimation bias in black African neonates.

Monte Carlo (MC) modeling is a widely used tool for investigating light-tissue interactions, optical device design, and validation of empirical measurements. MC modeling allows researchers the ability to simulate different physiological skin types, examples of which include white versus black pigmentation, different levels of skin hydration, and different levels of adipose content. Prior MC studies have demonstrated how different physiological skin types can impart influences on diffuse transcutaneous optical measurements; specifically, how TcB signals are impacted by increased skin pigmentation. These findings indicate that systematic compensation for pigmentation in the two techniques remains imperfect. Furthermore, quantification in both instances relies on development of calibration models.

Understanding how light incident on a strongly scattering medium, such as neonatal skin and biological tissue, interacts with the medium is of keen interest. The majority of current optical techniques are based on the elastic scattering phenomenon, which states that when light enters a medium the energy of a photon packet is preserved and no energy is transferred to the medium. In the case of the backscattered photons re-emerging from the tissue, there is a statistical correlation between mean penetration depth and offset distance between the emitter and detector of the system. The further away the detector is from the emitter, the deeper the light reaching the detector is likely to have gone into the medium, while also undergoing more absorption in the medium. However, the turbid nature of tissues and statistical nature of the relationship between photon pathlength depth and detector offset results in re-emerging photons at any given offset position having a mixed distribution of tissue pathlengths. Thus source-detector offset alone cannot be used to depth-segment optical signals from scattered photons.

Diffuse reflectance spectroscopy (DRS) has been used widely in the medical field to characterize tissue. DRS quantifies tissue structure and chemistry by measuring volume averaged optical properties, including the absorption coefficient, scattering coefficient, scattering anisotropy, and reduced scattering coefficient. DRS instrumentation comprised of light delivery to a tissue, and detection of a fraction of the reflected light from the tissue after the light has propagated and scattered within the tissue. Light scattering within tissues arises due to the refractive index difference at the interface between structures which have a higher refractive index than their surrounding intracellular fluid. Absorption in tissues occurs when a molecule is excited by an incident photon. Scattering and absorption tissue optical properties can be used to characterize tissue parameters such as bilirubin concentration, oxygen saturation, blood volume fraction, and beta-carotene concentration.

DRS has the advantage of simple, scalable, compact instrumentation with a demonstrated high accuracy characterization of both the optical absorption and the scattering properties of tissue. Other techniques are primarily sensitive to either scattering or absorption or contain complex optical components that are difficult to miniaturize.

A wide variety of DRS detection configurations and probes have been investigated. Illumination and light collection can be performed through bulk optics (i.e., lenses and filters) or fiber optics. Non-fiber probe systems typically include low-cost photodiode detectors (PDs) whose illumination-detection offset is fixed. Current designs include a limited number of up to three PDs due to size restrictions. Fiber probes can be comprised of small optical fiber bundles with one or more illumination and collection fibers. In their current form, fiber devices couple detection fibers to spectrometers to analyze diffuse reflectance primarily as a function of wavelength. Although detection fibers can be individually tracked to produce a fiber-by-fiber spatial map of different illumination and detection separations, this configuration requires an imaging spectrometer which dramatically increases device cost, complexity, and size. In addition, illumination and collection separations are fixed and finite.

In contrast, an approach that increases the dimensionality of the DRS data is spatially resolved diffuse reflectance (SRDR). SRDR uses multiple light collection locations with varied illumination and detection separations, enabling depth sensitivity because photons collected farther from the illumination location have traveled, on average, more deeply into the tissue. SRDR can use PDs for collection instead of fibers because PDs have higher numerical apertures and larger fill factors than fibers, and custom PDs can be fabricated in any shape. In summary, SRDR is a non-invasive optical technique used to sense biological changes at the cellular and sub-cellular level, and has the potential to significantly improve the current standard of care for such tasks as measuring physical parameters such as bilirubin.

A need remains for a system able to obtain information through the skin of a patient which is unbiased by optical variations such pigmentation, skin thickness, skin hydration, blood composition, cutaneous fat levels, and the like. A further need remains for a relatively low-cost, compact, non-invasive approach to measure physiological parameters such as bilirubin levels with performance comparable to blood tests and with less consumables. A still further need exists for a system that can better be applied for the systematic collection, analysis, and dissemination of medical information.

### SUMMARY OF THE DISCLOSURE

Some embodiments of the invention disclosed herein are set forth below, and any combination of these embodiments (or portions thereof) may be made to define another embodiment.

To meet these and other needs, and in view of its purposes, the present disclosure provides exemplary embodiments of systems and methods related to a 2D image sensor-based system for optical measurements of physiological parameters through the skin of a patient using spatially resolved diffuse reflectance. In some embodiments, the system includes a 2D image sensor adapted to be placed near or against the skin and having at least three wavelength-dependent spectral channels and a field of view adapted to capture multiple spatial regions of interest. The spectral channels can come from optical filters placed directly on or near a camera sensor, such as the red-blue-green Bayer filter typical in consumer color cameras, multiple narrow band optical illumination sources, a white light illumination source transmitted through custom filter sets, or a combination of these configurations, in optical design. At least one light source directs light onto the skin at a certain distance offset from the field of view. At least one detector receives light diffusely reflected from the skin and collects a 2D map of the entire reflectance spatial decay profile characteristic of the skin in across different spectral channels. In some embodiments, the final spatial channels used for analysis are extracted from the 2D sensor in software, and can be uniquely positioned per spectral channel and/or partially overlapping in each spectral channel. In some embodiment, a selector identifies multiple regions of interest in the 2D reflectance spatial decay map to serve as the final spatial channels as the elements of a multivariate regression model to approximate the physiological parameter of interest. The increased dimensionality of the system outputs results in improved quantification accuracy of the physiological parameters. The system also reduces bias in confounding optical factors.

Also provided are a related system and at least one computer-readable non-transitory storage media embodying software. The one or more computer-readable non-transitory storage media embodying software is operable when executed, in one embodiment, to perform a series of steps using the 2D image sensor system.

It is to be understood that both the foregoing general description and the following detailed description are exemplary, but are not restrictive, of the disclosure.

In one aspect, a transcutaneous spatially resolved diffuse reflectance optical measurement system, comprises a photodetector or multi-channel 2D sensor array, at least one light source, and a computing system communicatively connected to the photodetector and the light source, wherein the system is configured to perform wavelength-resolved measurements.

In one embodiment, the system further comprises a filter housing including at least one filter, wherein the filter housing is configured to be positioned between the light source, the photodetector and a skin sample to be measured, wherein the light source and the photodetector are positioned on a first side of the filter housing, and the skin sample to be measured is positioned on a second side of the filter housing opposite the first side.

In one embodiment, the computing system comprises a processor and a non-transitory computer-readable medium with instructions stored thereon, which when executed by the processor, perform steps comprising calibrating the system, measuring a skin sample via transcutaneous spatially resolved diffuse reflectance, calculating a bilirubin concentration withing the skin sample, and displaying the bilirubin concentration.

In one embodiment, the light source comprises a mobile phone flash LED, and the photodetector comprises a mobile phone camera.

In one embodiment, the filter housing further comprises an adaptor or a case suitable to connect to a mobile phone. In one embodiment, the filter housing is configured to align the at least one filter with the light source or photodetector.

In one embodiment, the at least one filter comprises first and second triple-band pass filters. In one embodiment, the first triple-band pass filter is positioned proximate to the light source, and the second triple-band pass filter is positioned proximate to the photodetector. In one embodiment, the first and second triple-band pass filters have identical spectral profiles.

In one embodiment, the light source is offset from a field of view of the photo detector in a range of 0 mm to 100 mm.

In one embodiment, the system is configured to measure in a spectral range of 300 nm to 2000 nm.

In one embodiment, the filter housing further comprises an angled channel configured to direct light from the light source to the field of view of the photodetector.

In one embodiment, light from the light source is filtered and enters the skin sample at a 15 degree to 45 degree angle with a 0 mm to 20 mm source-to-detector separation distance. In one embodiment, light from the light source is filtered and enters the skin sample at a 30 degree angle with a 0 mm source-to-detector separation distance.

In one embodiment, the filter housing comprises a highly opaque material configured to minimize light transmission through the material.

In one embodiment, the filter housing further comprises a pinhole of diameter 0.1 mm to 5 mm configured to reduce a half angle of the light source.

In another aspect, a transcutaneous spatially resolved diffuse reflectance optical measurement method to measure bilirubin concentration comprises providing the transcutaneous spatially resolved diffuse reflectance optical measurement system as described above, calibrating the system, measuring a skin sample via transcutaneous spatially resolved diffuse reflectance, calculating a bilirubin concentration withing the skin sample, and displaying the bilirubin concentration.

In one embodiment, the step of calibrating the system comprises measuring against one or more white or gray samples using a measurement sequence comprising obtaining a plurality of reflectance images with the light source on, and obtaining a plurality of background images with the light source off.

In one embodiment, the step of measuring a skin sample via transcutaneous spatially resolved diffuse reflectance comprises measuring the skin sample using a measurement sequence comprising obtaining a plurality reflectance images with the light source on, and obtaining a plurality of background images with the light source off.

In one embodiment, the step of calculating a bilirubin concentration withing the skin sample comprises converting a plurality of raw images to a plurality of digital negative images, demosaicing the plurality of digital negative images, identifying and removing outlier images from the demosaiced digital negative images, averaging the remaining demosaiced digital negative images to produce a skin reflectance image, a skin background image, a calibration reflectance image, and a calibration background image, subtracting the background images from corresponding reflectance images, normalizing the background subtracted skin reflectance image via the background subtracted calibration reflectance image to produce an RGB calibrated reflectance image, and applying a generalized linear model to obtain a bilirubin concentration.

In another aspect, a non-transitory computer readable medium comprising instructions which, when executed by a computer, causes the computer to perform steps comprising calibrating the system as described above, measuring a skin sample via transcutaneous spatially resolved diffuse reflectance, calculating a bilirubin concentration withing the skin sample, and displaying the bilirubin concentration.

### BRIEF DESCRIPTION OF THE DRAWING

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1(a) depicts a 3D physical model of an exemplary mobile phone TcB system according to the present disclosure which depicts illumination through the system, as well as into neonatal skin as illustrated with an MC model;

FIG. 1(b) illustrates the results of a MC simulation as a percent of reflectance measured based on source-detector offset;

FIG. 1(c) illustrates the results of a MC simulation as a fold change of reflectance in the red, green and blue filtered channels;

FIG. 2(a) illustrates a 3D-printed adapter which guides light towards skin and houses band-pass filters;

FIG. 2(b) illustrated the adapter as attached to a mobile phone case and aligned over the camera and flash;

FIG. 2(c) depicts a basic interface of a mobile phone application during image capturing, i.e., taking a measurement from the skin of a neonate;

FIG. 3(a) is a graph illustrating the demographic distributions of neonates included in study using the mobile phone TcB system;

FIG. 3(b) is a graph illustrating the distribution of TSB values obtained for all the neonates included in the study;

FIGS. 4(a), 4(b), and 4(c) show representative neonate RGB image data used for subsequent analysis, with FIG. 4(a) showing the red channel image with initial positions of the two regions-of-interest (ROI) overlaid, FIG. 4(b) showing the green channel image with initial positions of the three ROI overlaid, and FIG. 4(c) showing the blue channel image with initial positions of the four ROI overlaid;

FIG. 4(d) is a block diagram depicting model development and cross-validation;

FIGS. 5(a), 5(b), and 5(c) show the final mean ROI calculated across all cross-validations, with FIG. 5(a) showing the red channel image with the mean of final arc-shaped ROI which were generated due to the pattern search algorithm, FIG. 5(b) showing the green channel image with the mean of final ROI, and FIG. 5(c) showing the blue channel image with the mean of final ROI;

FIGS. 6(a) and 6(b) depict linear regression statistics, with FIG. 6(a) showing a correlation for the predicted TSB levels from the mobile phone TcB system against actual TSB and FIG. 6(b) showing the Bland-Altman plot of predicted bilirubin values versus TSB values;

FIG. 7 illustrates the overall process of generating neonatal skin Monte Carlo models, calibration, and testing;

FIGS. 8(a), 8(b), and 8(c) show the Monte Carlo model results for a high melanin test population when the calibration equation was generated using the representative population, with FIG. 8(a) showing that the bias is reduced with an increased number of wavelengths and detectors, FIG. 8(b) showing that the spread of LOA is reduced with an increased number of wavelengths and detectors, but the impact of the increase in the number of detectors is greater, and FIG. 8(c) showing that r² values also are increased with higher combinations of detectors and wavelengths;

FIG. 9(a) shows the r² value and FIG. 9(d) shows the spread of LOA when one ROI is used in each of the RGB channels;

FIG. 9(b) shows the r² value and FIG. 9(e) shows the spread of LOA when two ROI are used in each of the RGB channels;

FIG. 9(c) shows the r² value and FIG. 9(f) shows the spread of LOA when three ROI are used in each of the RGB channels;

FIG. 10 is a flow chart summarizing the image analysis;

FIG. 11 illustrates an example network environment associated with the disclosed mobile phone TcB system;

FIG. 12 illustrates an example computer system; and

FIG. 13 illustrates an example network environment.

FIG. 14 illustrates an example computing environment.

### DETAILED DESCRIPTION OF THE DISCLOSURE

In this specification and in the claims that follow, reference will be made to a number of terms which shall be defined to have the following meanings ascribed to them.

It is to be understood that the figures and descriptions of the present invention have been simplified to illustrate elements that are relevant for a clearer comprehension of the present invention, while eliminating, for the purpose of clarity, many other elements found in systems, devices and methods for spatially resolved diffuse reflectance for transcutaneous measurement. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The indefinite article “a” or “an” and its corresponding definite article “the” as used in this disclosure means at least one, or one or more, unless specified otherwise. “Include,” “includes,” “including,” “have,” “has,” “having,” comprise,” “comprises,” “comprising,” or like terms mean encompassing but not limited to, that is, inclusive and not exclusive.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “about” means those amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When a value is described to be about or about equal to a certain number, the value is within ±10% of the number. For example, a value that is about 10 refers to a value between 9 and 11, inclusive. When the term “about” is used in describing a value or an end-point of a range, the disclosure should be understood to include the specific value or end-point. Whether or not a numerical value or end-point of a range in the specification recites “about,” the numerical value or end-point of a range is intended to include two embodiments: one modified by “about” and one not modified by “about.” It will be further understood that the end-points of each of the ranges are significant both in relation to the other end-point and independently of the other end-point.

The term “about” further references all terms in the range unless otherwise stated. For example, about 1, 2, or 3 is equivalent to about 1, about 2, or about 3, and further comprises from about 1-3, from about 1-2, and from about 2-3. Specific and preferred values disclosed for components and steps, and ranges thereof, are for illustration only; they do not exclude other defined values or other values within defined ranges. The components and method steps of the disclosure include those having any value or any combination of the values, specific values, more specific values, and preferred values described.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems, devices and methods for spatially resolved diffuse reflectance for transcutaneous measurement.

This disclosure is directed to optical diagnostic devices and, more particularly, to systems for transcutaneous optical measurements that use spatially resolved diffuse reflectance.

A critical problem with all transcutaneous optical measurements is that they seek to obtain information through the skin which are unbiased by optical variations such pigmentation, skin thickness, skin hydration, blood composition, cutaneous fat levels, and the like. Established approaches including bilirubinometry rely on simplified approximations of light-tissue interactions, which do not offer complete removal of bias associated with optical variations. Recent studies have identified both race and ethnicity-dependent bias and decreased accuracy in transcutaneous measurements such as bilirubinometry. Clinical factors such as obesity, hydration, skin thickness, and others have also been associated with inferior measurement accuracy.

Focusing on bilirubinometry as an example, neonatal jaundice (NNJ) is characterized by the elevation of serum bilirubin levels driven by an underdeveloped bilirubin metabolism. NNJ is a normal condition clinically associated with some degree of yellowing in the skin and eyes that typically self-resolves in the first week after birth. Prolonged and extreme hyperbilirubinemia (EHB) is problematic, however, due to bilirubin's neurotoxicity and can ultimately lead to kernicterus, sever neurosensory deficit, and even death. A variety of factors, including genetic conditions prevalent in the populations of low- and middle-income countries (LMIC) such as glucose-6-phosphate dehydrogenase (G6PD) deficiency and immune mediated hemolytic diseases, exacerbate the problem in LMIC.

EHB-related neonatal mortality in LMIC is 638 infants per 10,000 births, compared to 3.7 infants per 10,000 births in high-income countries. Newborns in high-income countries are routinely screened for neonatal jaundice using TcB. TcB devices offer a widely used optical reflectance technique which performs measurements near bilirubin's absorption maxima at 460 nm along with at least one additional reference measurement above 500 nm to normalize for optical variations in tissue including perfusion, pigmentation, and scattering, while also detecting two distinct spatially offset detection channels with different depth-dependent biases. Over the last twenty years, TcB has become an important element in a systematic approach for screening a newborn's risk for EHB in high-income countries. As a result, appropriate treatments are administered in a timely fashion, and instances of chronic bilirubin encephalopathy (CBE) and EHB-related deaths are extremely rare in industrialized countries such as the United States.

Expanded adoption of TcB in LMIC has been proposed but the cost of currently available TcB devices remain an obstacle. In LMIC, the adoption of systematic approaches for screening, diagnosis, and therapy are not widespread mainly due to lack of resources. Although the gold-standard for measurement of bilirubin levels remains total serum bilirubin (TSB), in LMIC high rates of home birth and inconsistent availability of laboratory facilities and testing supplies remain a problem. Low-cost point-of-care blood testing solutions offer promise to reduce barriers to quantitative assessment of bilirubin in blood samples. A need remains, however, for a low-cost, non-invasive approach with comparable performance and less consumables.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention, and are not to be construed as limiting in any way the remainder of the disclosure.

To meet this need, the present disclosure teaches exemplary embodiments for a low-cost and mobile phone-based system that can be used to estimate TSB levels using SRDR. SRDR is a technique for performing the transcutaneous (TC) estimation of tissue chromophores with reduced influence from confounding parameters associated with tissue layers adjacent to the layer where the chromophores of interest reside. In contrast with prior point-of-care devices, SRDR images diffuse reflectance point-spread functions with a 2D camera module instead of the reflectance at a finite set of positions using single-point detectors. In one embodiment, the mobile phone-based TcB system offers a strong potential for integration with communications networks for coordination of care, healthcare data management, and real-time patient monitoring. The system mimics clinical TcB measurements by directly measuring a spatially offset diffuse reflectance in direct contact with the infants' skin.

Monte-Carlo (MC) modeling of photon migration in tissue has been a well-suited approach for theoretical investigation of diffuse reflectance in TcB, and has provided valuable insights into the influence of pigment, scattering, illumination and collection geometry, and light source selection. In the disclosed system, MC models of reflectance from neonatal skin are used to guide the design of an adapter for filtered Red-Green-Blue (RGB) mobile phone camera reflectance measurements. A study was performed across two sites in the United States and Nigeria in order to include neonates with a wide range of pigmentation, and a generalized linear model was developed using multiple spatial offset regions-of-interest (ROI) across the RGB reflectance channels. Results indicate that the mobile phone-based system accurately estimates bilirubin in neonates.

In some embodiments, the 2D camera module used in the mobile phone TcB system can be any 2D image sensor. For example, CMOS (complementary metal oxide semiconductor) and CCD (charge coupled device) imagers are suitable. CMOS sensors have high speed, low sensitivity, and high, fixed-pattern noise. Just like a CMOS sensor, a CCD sensor converts light into electrons. Unlike a CMOS sensor, a CCD sensor is an analog device having a silicon chip that contains an array of photosensitive sites. Also suitable are versions of a detector array, including different hardware embodiments such as InGaAs arrays sensitive in the infrared spectral band.

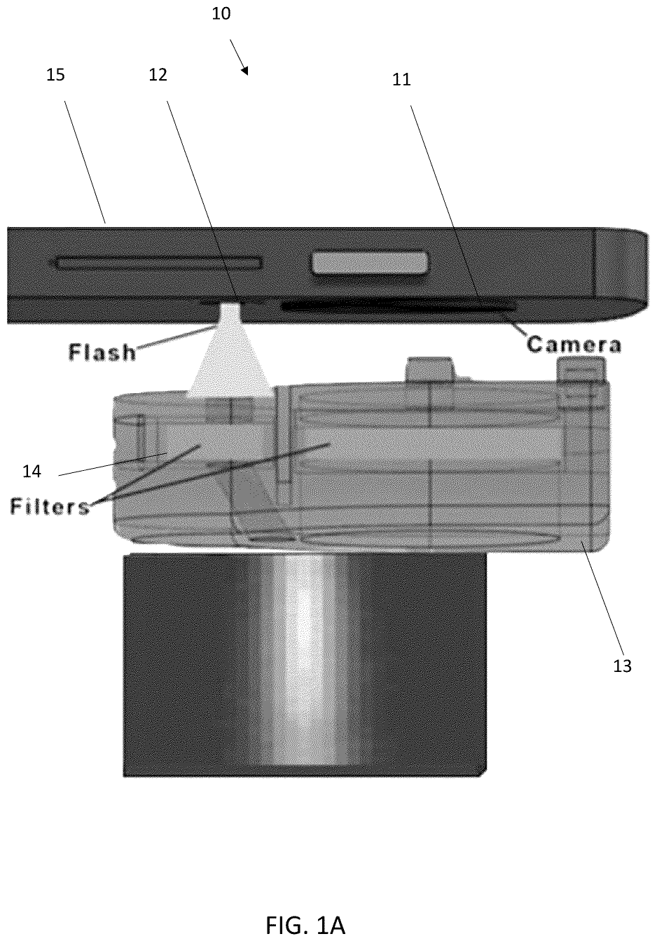

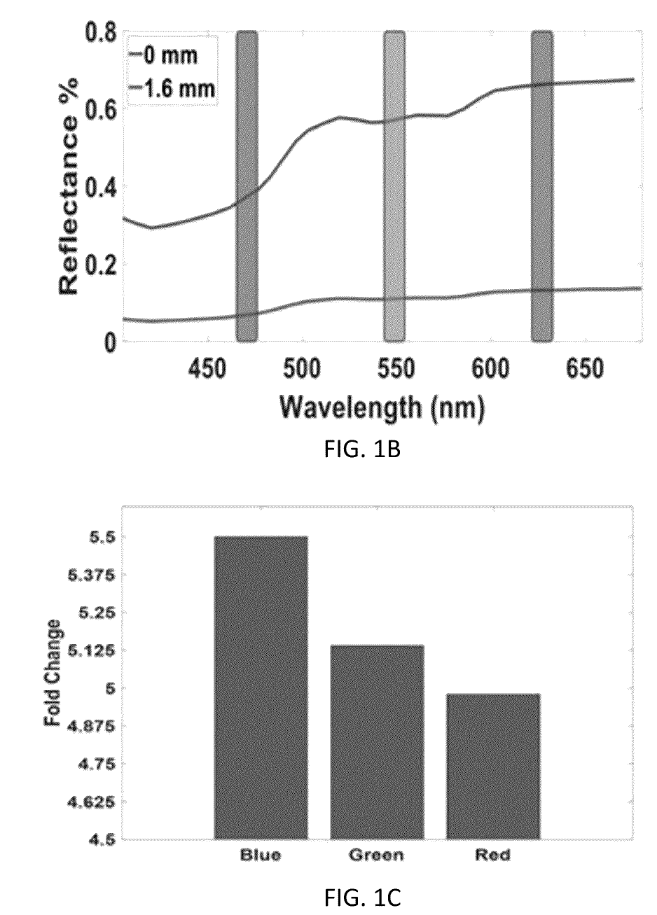

In one embodiment, the system includes an adaptor that can be placed over the light-emitting diode (LED) and camera of a mobile phone to specify illumination and collection geometry and optimize collection of the wide range of reflectance intensities observed when capturing spatially resolved images of spatially offset reflectance seen across the 400-700 nm spectral range. Preliminary data suggested a large mismatch in reflectance across the blue and red channels leading to an insufficient blue channel reflectance signal at appropriate spatial offsets. MC models were created of diffuse reflectance from neonatal skin with illumination and collection parameters that matched the adaptor and simulation outputs were used to determine the relative benefit of a reduction in source-detector separation to reduce red/blue signal disparities and to simplify measurement. A prototype smartphone adaptor, along with a representative MC simulation of photon flux in the skin, is shown in FIG. 1(a) More specifically, FIG. 1(a) depicts a 3D physical model of the mobile phone TcB system which depicts illumination through the system, as well as into neonatal skin as illustrated with an MC model. In some embodiments, a transcutaneous spatially resolved diffuse reflectance optical measurement system 10 comprises a photodetector 11, a light source 12, a filter housing 13 including at least one filter 14, and a computing system 15 communicatively connected to the photodetector and/or the light source. In some embodiments the filter housing is configured to be positioned between the light source, the photodetector and a skin sample to be measured, wherein the light source and the photodetector are positioned on a first side of the filter housing, and the skin sample to be measured is positioned on a second side of the filter housing opposite the first side.

FIG. 1(b) illustrates the results of a MC simulation as a percent of reflectance measured based on source-detector offset. FIG. 1(c) illustrates the results of a MC simulation as a fold change of reflectance in the red, green and blue filtered channels.

MC modeling was performed using the Monte Carlo eXtreme (MCX) platform. A generalized tissue optical model was configured to simulate neonatal skin, with parameters related to skin thickness, scattering, and chromophore concentration and extinction. Melanosome fractional volume was set at 10% to determine model performance in darkly pigmented skin, where overall signal intensities are lower. The optical illumination and detection configuration in MCX was directly informed by both the physical and optical specifications of the LG Nexus 5 Android smartphone and the adaptor, including LED-to-camera offset, illumination spot size, detector size, and imaging optics. Two exemplary optical configurations were explored: (1) the offset between the center of the illumination beam and the proximal edge of the camera field-of-view was set to 1.6 mm, and (2) the offset was reduced to 0 mm. In both configurations, the adaptor thickness was set to 10 mm, and modeled spectral diffuse reflectance from 400-700 nm was summed across approximately a 9 mm×12 mm field of view, which was subsequently verified through empirical measurements with a microscopy calibration grid. After running the MC simulations, modeled spectral diffuse reflectances were compared between the two spatial offset configurations (FIG. 1(b)), and the fold-change differences in spectral intensities were compared across each RGB color channel (FIG. 1(c)). The results of these simulations indicate differences in the relative magnitude of color-channel dependent changes in detected reflectance for the two flash-detector offsets, and inform any potential reduction in the range of reflectance values measured across channels.

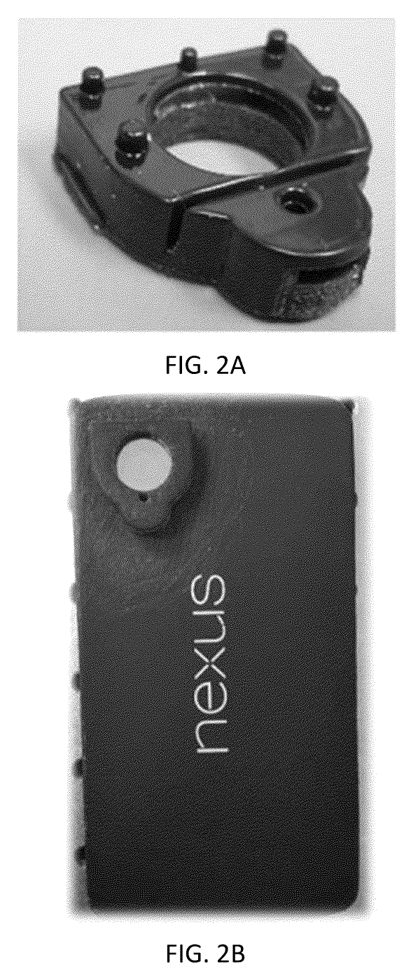

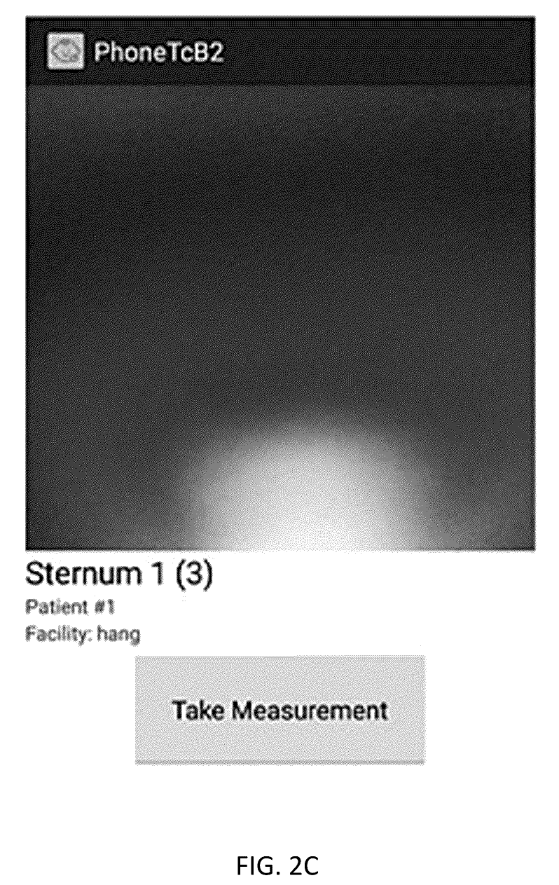

Based on the results of the MC models, the exemplary adaptor shown in FIG. 2(a) was developed to be placed between the infant's skin and the camera and LED of a mobile phone (e.g., a LG Nexus 5 smartphone). A purpose of such placement is to redirect LED illumination to a confined spot with minimal spatial offset from the camera's field of view. In some embodiments, the adapter is 3D printed (using a Connex 3 Object 500 3D printer, for example, available from Stratsys Ltd. of Minnesota) and has a thickness of 10 mm. The adapter is made of black material (a Vero Black material, available from Stratsys Ltd., is suitable) in order to reduce the intensity of light that may transmit through the plastic. As best illustrated in FIG. 1(a), the adapter houses triple-band pass optical filters for improved spectral isolation of RGB measurements. In other exemplary embodiments, an adaptor similar to that shown in FIG. 2(a) can be configured for use with any suitable devices and systems including for example a handheld device, a desktop device in a clinical setting, or other similar devices.

In the embodiment shown, the adapter is attached (e.g., fixed) to a generic snap-on protective mobile phone case, and aligned over the camera and flash LED, as illustrated in FIG. 2(b). A 2 mm pinhole on the phone-facing side of the adapter reduces the half angle of the LED from approximately 41 degrees to 21 degrees to preserve the spectral performance of a triple-band pass filter (the Idex Semrock FF01 filter with D=5 mm, available from Semrock, Inc. of Illinois, is suitable) with passbands at 474±10 nm (blue), 554±10 nm (green), and 635±10 nm (red). After the filter, an angled 2 mm channel directs the light towards the camera field of view, with a minimum offset that is limited by the performance of the 3D printer. The spatially offset diffuse reflectance is then collected via a separate optical path that includes a notched space for a second, larger, triple-band pass filter (D=10 mm) with an identical spectral profile.

In some embodiments, a custom mobile application configures mobile-phone image acquisition and guides users through calibration and clinical measurements. The basic interface of the mobile phone application during image capturing (i.e., taking a measurements from the skin of a neonate or patient) is presented in FIG. 2(c). Image data is configured to be saved without pre-processing or compression in raw 16-bit format in order to ensure the data could be used for quantitative measurement of reflectance. Camera acquisition parameters were fixed, including focus, exposure time, gain, and ISO. (ISO refers to a camera setting, i.e., a number, that establishes the sensitivity of the camera to light. The higher the ISO, the more sensitive the camera sensor becomes and the brighter its photographs appear.) In one example experiment, each measurement included an acquisition of three successive tissue reflectance images with an exposure time of 400 ms and an ISO of 4,500, along with a paired set of three dark background images collected with the LED off. In the studies that were conducted, three replicate sets of measurements were collected from each infant to ensure unexpected clinical scenarios such as sudden movements did not reduce enrollment yield.

In one example experiment, calibration was performed by measurement of a custom calibration standard comprising of a 25×25×5 (thick) mm, high-density polyethylene (HDPE), white, plastic block housed in a Vero Black casing using an identical sequence of reflectance (LED on) and background (LED off) image acquisitions. Paired calibration measurements were collected for each patient enrolled.

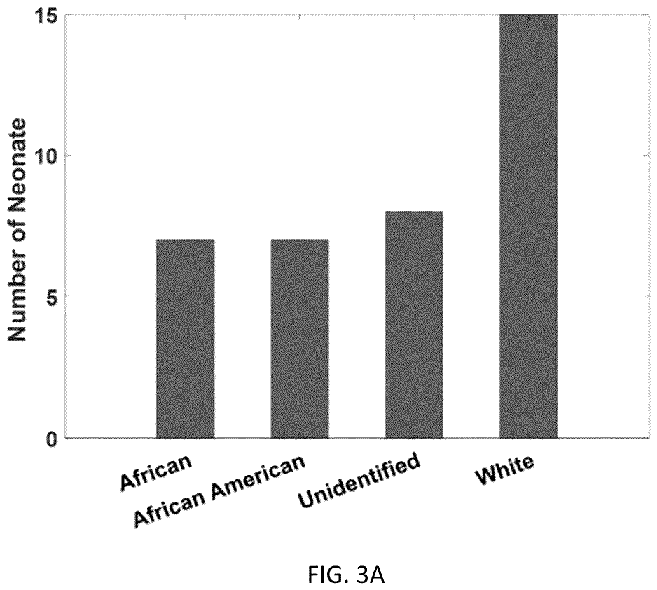

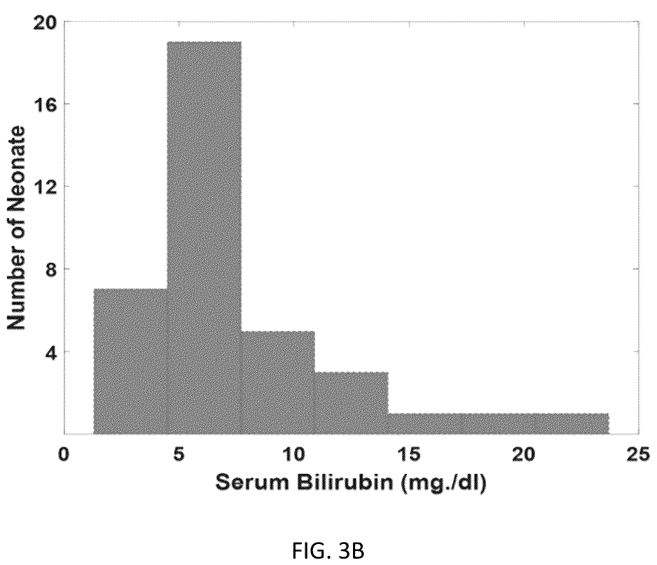

Measurements were collected using the mobile phone TcB system from thirty-seven neonates (thirty from Vanderbilt University Medical Center (VUMC) in Nashville, TN, USA, and seven neonates from Aminu Kano Teaching Hospital (AKTH) in Kano, Nigeria. Healthy infants between 24-72 hours postnatal age were considered eligible for this study. Exclusion criteria included infants born with known or estimated gestational age<32 weeks, weighed under 1,500 grams, or prior phototherapy. Each mobile phone measurement was obtained within 30 minutes of a blood sample used to obtain TSB. Written consent was obtained from all parents, and all studies were performed in accordance with the institution's human subjects research approvals (VUMC IRB130471, AKTH EC1390). All measurements were collected using the smartphone in direct contact with the sternum of the neonates while in rooms where bright ambient lighting was minimized. The demographic distributions of the neonates are provided in FIG. 3(a). There are fourteen African or African American neonates, fifteen Caucasian neonates, and eight race-unclassified neonates. The distribution of TSB values obtained for all the neonates are shown in FIG. 3(b), indicating a right-skewed, non-normal distribution characteristic of newborn TSB, with a majority of values between 5-10 mg/dL, and some neonates with elevated values as high as 23.7 mg/dL.

Raw images were extracted from the mobile phone TcB system and converted to digital negative (.dng) format with an Adobe DNG Converter prior to import into the MATLAB® computing environment. MATLAB is an abbreviation for “MATrix LABoratory” and is the registered trademark for a proprietary multi-paradigm programming language and numeric computing environment developed by The MathWorks, Inc. of Natick, Massachusetts. The MATLAB environment allows matrix manipulations, plotting of functions and data, implementation of algorithms, creation of user interfaces, and interfacing with programs written in other languages.

The DNG images were demosaiced. A demosaicing (also called de-mosaicing, demosaicking, or debayering) algorithm is a digital image process used to reconstruct a full color image from the incomplete color samples output from an image sensor overlaid with a color filter array (CFA). The algorithm is also known as CFA interpolation or color reconstruction. Many modern digital cameras can save images in a raw format allowing the user to demosaic them using software, rather than using the camera's built-in firmware. The demosaicing algorithm results in red, green, and blue (RGB) images that are resized to a quarter of the original image to reduce the data size for analysis.

In the example experiment, all images were processed through a multivariate k-nearest-means outlier detection algorithm which was used to identify sets of images which contained poorly performed measurements (i.e., infant motion, device non-contact), but any suitable image processing algorithms can be utilized. Once the outliers were removed, each set of images (sternum and calibration images) was averaged resulting in one final image each for sternum reflectance, sternum background, calibration reflectance, and calibration background associated with each neonate. Reflectance images were background subtracted and normalized by calibration images in order to produce a single RGB calibrated reflectance image for each neonate.

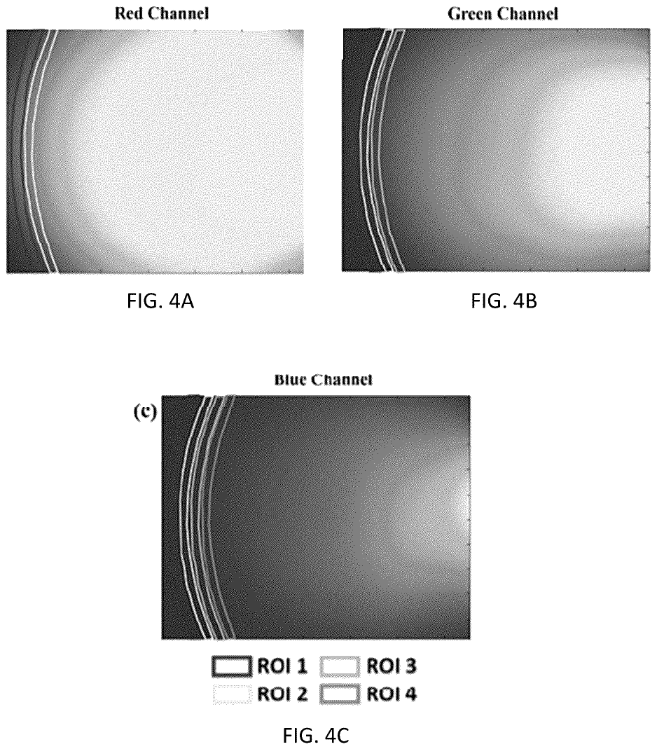

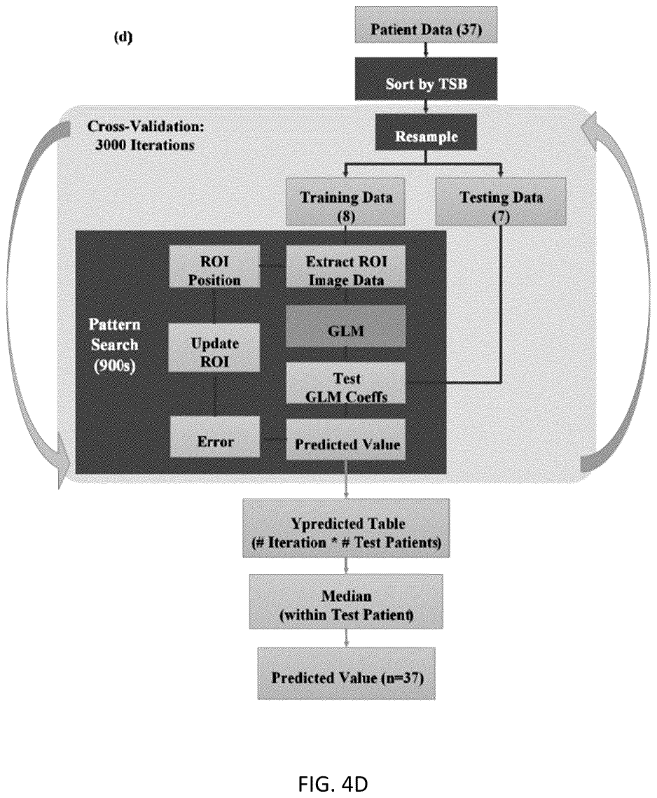

FIGS. 4(a), 4(b), and 4(c) show representative neonate RGB image data used for subsequent analysis. More specifically, FIG. 4(a) shows the red channel image with initial positions of the two regions-of-interest (ROI) overlaid. FIG. 4(b) shows the green channel image with initial positions of the three ROI overlaid. FIG. 4(c) shows the blue channel image with initial positions of the four ROI overlaid.

Optical estimation of bilirubin levels is classically informed through creation of linear regression models of the reflectance from unique spectral and spatial channels against matched TSB values. In the mobile phone TcB system, the pre-processed images represent a spatially resolved map of tissue diffuse reflectance, and thus a linear model can be created similar to classical TcB using multiple unique spatial offset ROI within each RGB spectral image channel. Unlike conventional TcB, where physical detectors placed at unique offsets define independent spatial channels, collection of images of diffuse reflectance produces a spatially resolved reflectance map and offers the opportunity to define multiple spatial offset channels uniquely in each RGB spectral channel. In order to determine appropriate placement of the spatial offset channels and to develop the corresponding linear model, a data-driven approach was used to train and cross-validate a bounded pattern search algorithm which optimized placement of specific spatial offset channel ROI for use in a generalized linear model (GLM) to predict TSB.

FIG. 4(d) is a block diagram depicting model development and cross-validation. The overview of the image analysis using a pattern search algorithm is presented. Input, intermediate, and output data are depicted as orange boxes, primary operations as purple boxes, and secondary operations as green boxes.

Based on the right-skewed non-negative distribution of the TSB values illustrated in FIG. 3(b) and the resulting limited number of neonates enrolled with TSB values>15 mg/dL, randomized selection of neonates to include in training subsets would result in a high likelihood that models constructed in individual cross-validation iterations were not informed with data across the full clinically relevant range from 0 -20 mg/dL of TSB. In order to account for this enrollment-dependent limitation in the example experiment, the full data set was first sorted according to TSB values, and then divided into four different ranges: 0-5 mg/dl, 5.01-10 mg/dl, 10.01-15 mg/dl, and 15.01-25 mg/dl. Training data sets included randomized resampling of two neonates each across the four TSB bins to total eight uniformly sampled neonates across the full TSB range. The testing sets were then selected from the remaining pool of neonates with two neonates from the first three ranges and the one remaining neonate left in the highest TSB range (only three enrollees had TSB levels >15 mg/dL) to total seven neonates. Cross-validation using this approach for resampling training and testing data over many iterations then produced a composite estimate of the performance of the model.

In the example experiment, a bounded pattern search algorithm implemented in the MATLAB optimization toolbox was used to optimize ROI placement for the linear model, however any suitable algorithms and cross validation routines can be utilized. Here, the spatial offset areas are arc-shaped ROI in each spectral (RGB) image frame defined based on their position, radius, and width. Initial conditions for two ROI in the red channel, three ROI in the green channel, and four ROI in the blue channel are shown in FIGS. 4(a), 4(b), and 4(c), respectively. The pattern search algorithm extracts mean values within the ROI as the observed variables for a gamma distribution (non-negative constraint) based GLM to predict TSB. The pattern search algorithm aims to iteratively adjust the positions of the ROI in order to optimize the sum square error of the GLM over a duration of 900 seconds. After completion of the pattern search, the predicted values of the test set were stored in a table, and the training and testing routine was cross-validated over 3,000 iterations, resulting in 21,000 estimates (about 550 per neonate). The median predicted value for each set of predictions for an individual neonate was then determined to be the final predicted neonate value. A correlation plot for predicted bilirubin values versus TSB and a Bland-Altman plot (a diagnostic tool that provides a useful display of the relationship between two paired variables using the same scale) were generated to report model performance, bias, and limits of agreement. In addition, the final ROI parameters were calculated as the mean positions across all cross-validations.

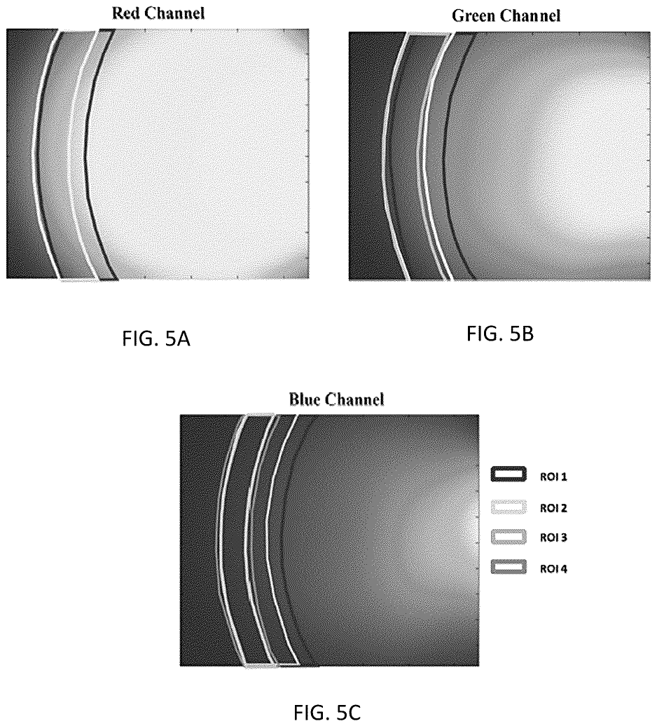

FIGS. 5(a), 5(b), and 5(c) show the final mean ROI calculated across all cross-validations. More specifically, FIG. 5(a) shows the red channel image with the mean of final arc-shaped ROI which were generated due to the pattern search algorithm. FIG. 5(b) shows the green channel image with the mean of final ROI. FIG. 5(c) shows the blue channel image with the mean of final ROI.

A MCX model was used to compare the effect of reducing the illumination-detector offset on the collected diffuse reflectance signal from bilirubin. FIGS. 1(b) and 1(c) represent the impact of source detector offset on diffuse reflectance intensities across the spectral range. In FIG. 1(b), there is a clear increase in measured reflectance when the source-to-detector separation is decreased from 1.6 mm to 0 mm. In addition, the modeled spectra indicate blue channel reflectance to be the lowest, while red channel reflectance is the highest. FIG. 1(c) shows a fold increase in signal between the two simulated spectra for the decreased offset. Compressing the range of reflectance across the channels would thus correspond to a larger fold change in the blue channel versus the red channel, which is seen in the data, as the blue channel of the RGB signal is increased by 5.5 times while the red channel is increased by 4.97 times when the source-to-detector separation is 0 mm versus 1.6 mm. Thus, decreasing the offset both increases blue channel reflectance and narrows the different signal intensities between red and blue channels, which results in improved exposures across all RGB channels.

For the example experiment, the TcB system illustrated in FIG. 1(a) and FIG. 2(b) was directly mounted over the flash and camera of the LG Nexus 5 mobile phone. Along with the adapter, caretakers use the mobile phone TcB app to capture images of the skin of the neonates. The light from the flash is then filtered and enters the skin at a 30 degree angle with a 0 mm source-to-detector separation.

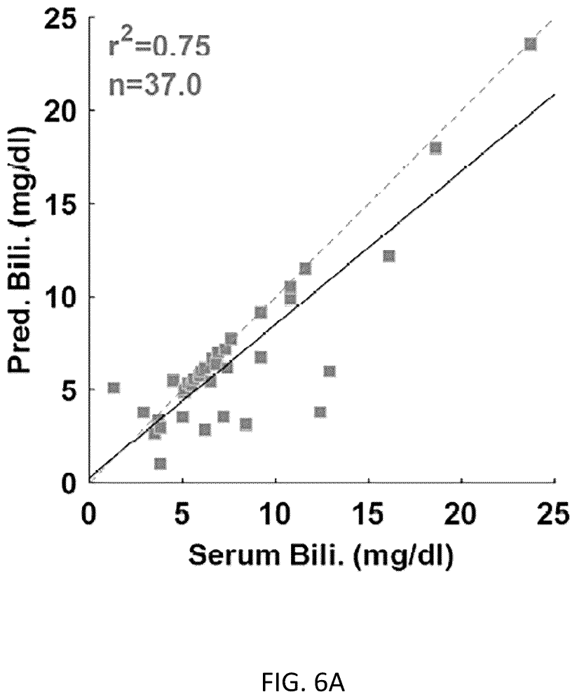

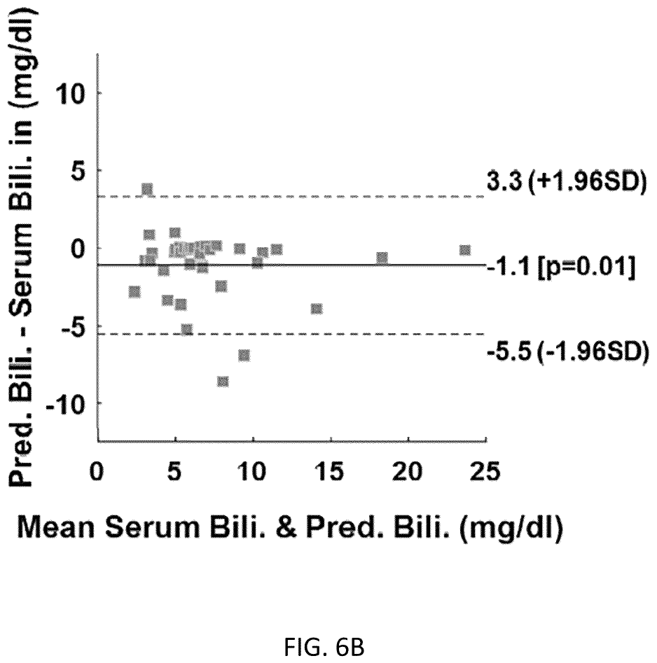

The ability to measure bilirubin levels was evaluated in the study conducted at VUMC and AKTH. FIG. 3(a) represents the demographic breakdown of the thirty-seven neonates that participated in this study. TSB and mobile phone TcB system measurements were collected from each neonate. For the example experiment, the measured bilirubin TSB levels complemented the clinical bilirubin range from 0-25 mg/dL, with the majority ranging between 5 and 10 mg/dL. FIG. 6(a) shows a correlation for the predicted TSB levels from the mobile phone TcB system against actual TSB with an r² value of 0.75. The Bland-Altman plot in FIG. 6(b) of predicted bilirubin values versus TSB values shows a mean difference of −1.1 mg/dL between the mobile phone TcB system and TSB measurements, and 95% limits of agreement (LOA) are 3.3 mg/dL to −5.5 mg/dL (range between the upper and lower limits of LOA equal to 8.8 mg/dl). These results indicate the clinical ability of the mobile phone TcB system to accurately estimate TSB values in a racially diverse group of neonates.

For the example experiment, the root-mean-square errors (RMSE) for three different skin groups (light or dark skin) were calculated from the resulted predicted values. In light-skinned neonates (n=15), the RMSE was 3.38 mg/dL; in dark-skinned neonates (n=14), the RMSE was 1.95 mg/dL; and in the neonates with an unclassified skin color, the RMSE was 0.55 mg/dL. It was also found that the mobile phone TcB system over-estimated TSB levels in 20% of cases for light-skinned neonates compared to 35.7% of cases for dark-skinned neonates. Importantly, all over-estimated TcB values in the dark-skinned neonates were less than 0.9 mg/dL.

EHB contributes up to 14% of neonatal deaths and causes a significant amount of neurological sequelae in low-resource settings. Reducing the global burden of EHB in LMIC is a complex problem, including challenges in the lack of genetic screening, maternal education, adoption of universal practices for newborn screening, and expansion of availability of phototherapy. The low-cost mobile phone TcB system takes advantage of the widespread and expanding access to smartphones across low-resource and rural areas in LMIC and has substantial potential to improve screening practices. Moreover, the connection of data and information services through smartphones offers the potential for coordination of treatment services for jaundiced infants requiring care in remote areas.

The disclosed mobile phone TcB system for TcB measurement uses an optical adapter fitting commercially available protective mobile phone cases. The adaptor enables collection of spatially resolved images of diffuse reflectance with an offset illumination spot through a set of optical filters. The filters used in the adaptor are scientific-grade coated glass substrate, low-cost, durable, optical filters that allow production of adaptors for <$50. The use of a passive adaptor, in combination with the novel approach of extracting multiple spatial and spectral ROI reflectance channels within the RGB image data, offers an improved approach for TSB estimation in low-resource settings.

As shown via the example experiment, the disclosed mobile phone TcB system estimated TSB within 3.3 to −5.5 mg/dL, with a bias of −1.1 mg/dL. See FIG. 6(b). These results are comparable to previously published results of TcB-TSB agreement in large, multi-ethnic study populations, which have a maximum span of the reported LOA falling between approximately 4 and −6.0 mg/dL and biases between 1.6 and −0.8 mg/dL. These data support use of the disclosed mobile phone TcB system as a TcB surrogate.

An important issue reported in clinical TcB devices is both increased error and a systematic overestimation bias in black African neonates (LOA of 6.7 to −0.65 mg/dL, bias of 3.04). TcB nomograms constructed using only black African neonates have been shown to address these issues, indicating these problems may arise from skewed racial composition in enrollment of subjects used during construction of the TcB regression models. Enrolment in the study using the disclosed mobile phone TcB system was nearly even between those of African-origin and white infants in an attempt to ensure that enrollment biases minimally contribute to model construction. Although the overall bias of the study is slightly negative, specific evaluation of black and African neonates indicates opposite trends in comparison with clinical TcB, where 37% (5 out of 14) of the black neonates exhibited over-estimation, but the amount of over-estimation in all of these cases was lower than 0.9 mg/dL (mean over-estimation =0.23 mg/dL). It was also found that the mean-underestimation in black and African neonates was 1.74 mg/dL. It is worthwhile noting the results from the study do not indicate increased error of the over-estimation bias previously seen using TcB for black and African neonates.

A fundamental aspect of using the disclosed mobile phone TcB system lies in the extraction of multiple individual spectral and spatial offset ROI from filtered RGB images of spatially offset diffuse reflectance. Although clinical approaches to TcB measure diffuse reflectance in up to two different fixed spatial offset channels using between two and five wavelength channels to create a model for bilirubin estimation, the mobile phone TcB system uses nine uniquely optimized spatial ROI across three different wavelength channels to inform a GLM.

Selection of an appropriate acquisition time that would allow proper exposure of the spatial decay of spatially offset diffuse reflectance across the RGB color channels represented a challenge for measurement configuration. The disclosed mobile phone TcB system requires configuring camera hardware to collect the raw, uncompressed, 16-bit output of the detector array, as opposed to a JPEG compression, in order to retain the ability to perform absolute quantification of reflectance and preserve the available dynamic range of the detector. The more than two-fold difference in the red channel-to-blue channel reflectance coupled with the requirement for large, uncompressed raw image data motivated selection of a single exposure time which served as a compromise allowing collection of maximal signal in the blue channel while still retaining a limited range of un-saturated reflectance in the red channel. MC models of reflectance in neonatal skin were used to investigate the benefit of altering source-detector offset in order to increase blue channel reflectance. Alternative strategies such as collection of multiple raw images at different exposure times for high-dynamic range compression represents a strategy which may improve the spatial range of unsaturated data available to place ROI. Although this strategy may improve the TSB prediction model, it would require collecting large file size datasets.

The cost of currently available TcB devices remains an obstacle for their adoption within low resource settings and LMIC as part of standard practice. The results achieved by the disclosed mobile phone TcB system, using a simple, low-cost adaptor and unique algorithm for extraction of multiple spatial and spectral ROI from images of spatially offset reflectance, show that the system is effective in estimating TSB to within an acceptable range and may prove valuable in LMIC settings.

A further study was conducted using the disclosed mobile phone TcB system. In this study, MC modeling was applied to better understand and develop a method to correct for the influence of pigmentation. A MC-based simulations platform was developed to predict bilirubin. The platform simulated different racial populations as characterized by unique fractional volume of melanosome distributions. In addition, the MC detection framework was configured with multiple-source detector (SD) offset regions. The simulation results showed that having multiple SD offsets in multiple wavelength channels could reduce the bias and error range in high melanin testing sets. Finally, multiple spectral and spatial detector channel analysis in clinical mobile phone TcB data found a higher number of spatial detector channels improved TcB accuracy and reduced bias in a high melanin population testing set.

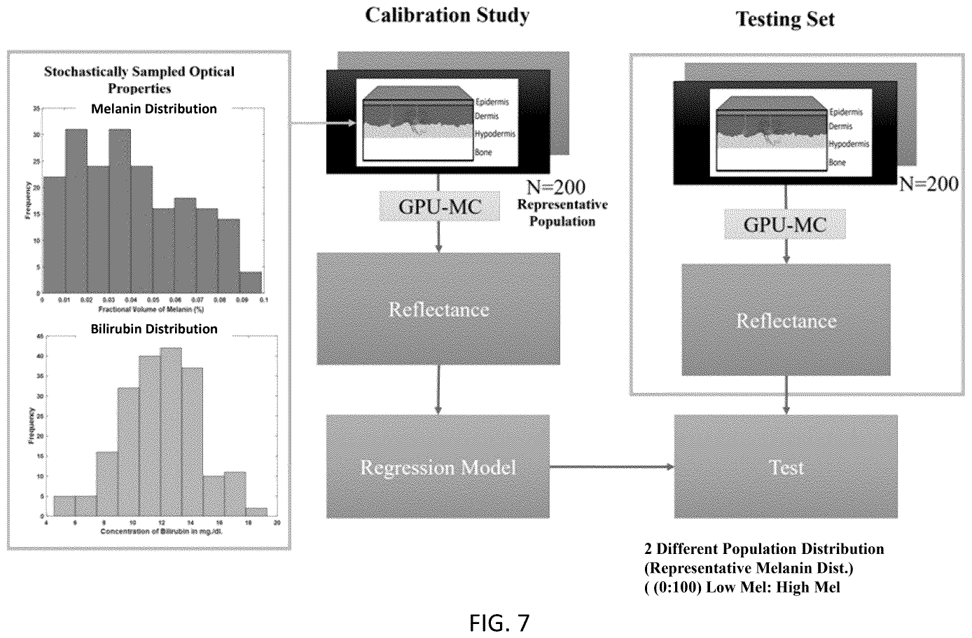

In this study, multiple theoretical bilirubinometer calibration models and detection channels were generated using MC simulations. The overall process of generating neonatal skin models, calibration, and testing is depicted in FIG. 7. Each finger model was generated with optical properties of epidermis and dermis statistically sampled from three different mixtures of racial groups. The finger models of FIG. 7 were generated using BioRender, an online software tool that helps scientists to quickly create and share figures. A calibration study was used to create regression models from a representative population distribution with 20% dark-skinned neonates. The regression model was used to test two different testing population samples.

A four-layer skin model mimicking the neonatal skin was created. The absorption coefficient and scattering coefficients of epidermis and dermis were statistically sampled from probability distributions with specific mean values and ranges. The optical properties of all other layers were configured with fixed properties. Importantly, two unique distributions for the fractional volume of melanosome were used to inform the epidermis, with different mean values, in order to model different racial distributions.

Three cohorts with different racial mixtures were generated. First, a calibration study population set was generated using a specific demographic distribution. The other two population samples were based on all high melanin and representative melanin distributions which were used as a testing set. For each of the population sets, a total of n=200 skin models were generated.

To simulate TcB-like devices and predict the bilirubin using MC simulations, one broadband LED-like source was modeled at five different wavelengths (450 nm, 500 nm, 550 nm, 600 nm, and 650nm) and five detectors spatially separated from the source by 0.5 mm, 1 mm, 2 mm, 3 mm, and 4 mm. The wavelengths and spatial offsets were selected so that they are in the visible region and similar to those previously discussed above. Thus, five different source-detector offsets were developed in the model space. The simulations were then carried out using MCXLAB software.

A MEX file is a type of computer file that provides an interface between MATLAB or Octave and functions written in C, C++, or Fortran. “MEC” stands for “MATLAB executable.” When compiled, MEX files are dynamically loaded and allow external functions to be invoked from within MATLAB or Octave as if they were built-in functions.

MCXLAB is the native MEX version of MCX for MATLAB and GNU Octave software. MCXLAB compiles the entire MCX code into a MEX function which can be called directly inside MATLAB or Octave. The input and output files in MCX are replaced by convenient in-memory struct variables in MCXLAB, thus, making it much easier to use and interact. MATLAB/Octave also provides convenient plotting and data analysis functions. With MCXLAB, an analysis can be streamlined and sped-up without involving disk files.

MC simulations of 200 neonates were performed to developed calibration model equations. Because multiple combinations of source-detector offsets and wavelengths were used, a total set of twenty-five different calibration equations were generated for the calibration study based on the combination of detector and wavelength numbers. A similar strategy was applied to generate MC simulations of two different testing sets.

Calibration equations for multiple spatially resolved detectors (i.e., ROI within the image sensor) across three different wavelength channels within mobile-phone TcB images were generated according the following procedure. Using the disclosed mobile phone TcB system, smartphone images were acquired from seventy-one neonates from two different centers (VUMC and AKTH). Thirty-seven neonates were enrolled in a multi-ethnic cohort for the calibration equation generation. These thirty-seven neonates had a mixture of ethnic backgrounds (fourteen African or African American, eight race unidentified, and fifteen white neonates). In these thirty-seven neonates, the TSB (mg/dl) distributions were mostly concentrated around the 5-10 mg/dl range which could have impacted the calibration equation to predict bilirubin. A resampling technique was applied to make the bilirubin range uniform which ensured bilirubin values are uniformly sampled from four different ranges (0-5 mg/dl, 5.01-10 mg/dl, 10.01-15 mg/dl, 15.01-25 mg/dl). From each of the ranges, two neonates (total eight neonates) were taken for calibration equation generation. For the testing set, the remaining thirty-three neonates were selected. All the neonates in this testing set had darker skin which is comparable to an all high melanin test set. From these thirty-three neonates, using the uniform resampling techniques, eight test neonates were selected from four different ranges (5-10 mg/dl, 10.01-15 mg/dl, 15.01-20 mg/dl, 20.01-25 mg/dl).

To extract the information from the neonatal images, arc-shaped ROI were defined using a fixed center and an inner and outer diameter. For the initial model, one ROI was placed in each of the color channels (red, green, and blue). The coefficients from the calibration set were used to generate a normal distribution-based generalized linear model. Then, the model was used to predict the bilirubin values. To measure the error, the absolute sum of error was calculated. A pattern search algorithm from MATLAB was used to move the ROI so that the testing error is reduced. The pattern search for each iteration was allowed to run for 120 seconds. A total of 2,000 iterations of resampling were carried out to generate a distribution of predicted values for each neonate. Later, the modes of these distributions were taken to predict the bilirubin values. To get the modes, the predicted values were rounded to the nearest quarter that could include a maximum inherent error of ±0.25 mg/dl in the predicted values. The r² values and the LOA spread were also calculated.

In statistics, the coefficient of determination, denoted R² or r² and pronounced “R squared,” is the proportion of the variation in the dependent variable that is predictable from the independent variable or variables. R-squared is a statistical measure of fit that indicates how much variation of a dependent variable is explained by the independent variable or variables in a regression model.

Finally, this multiple spatial offset ROI-based pattern search process was expanded to two and three ROI in each color channel. This approach of using multiple ROI presented a unique opportunity to use multiple source-detector offsets to predict bilirubin from smartphone TcB images.

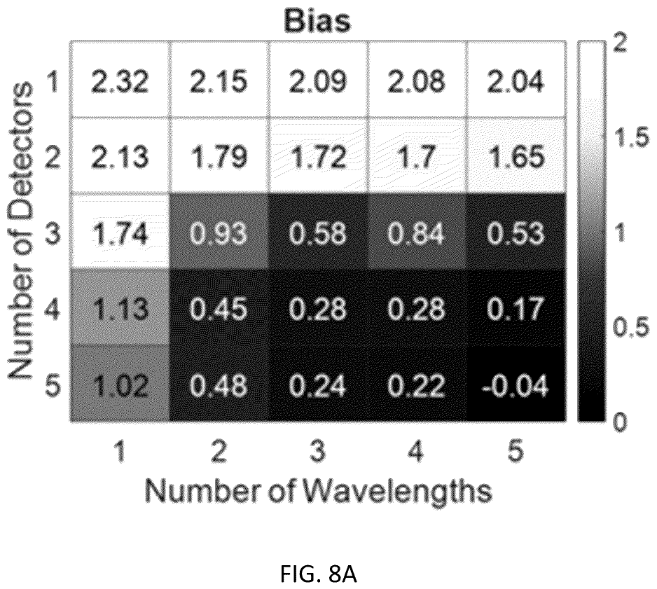

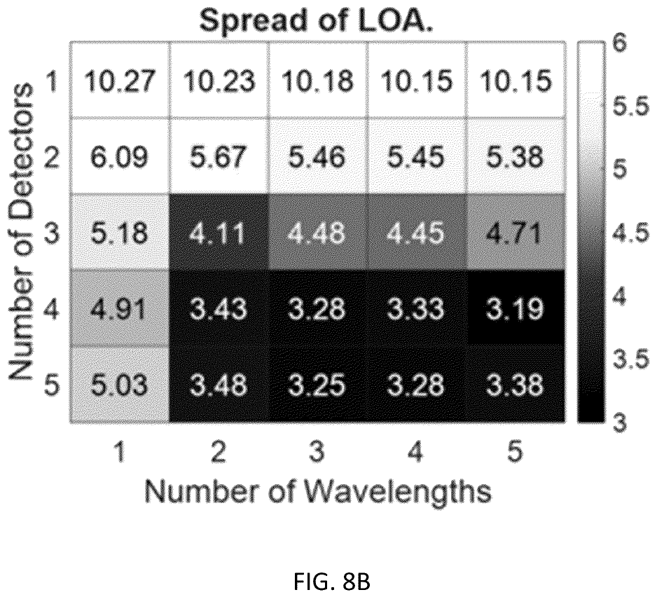

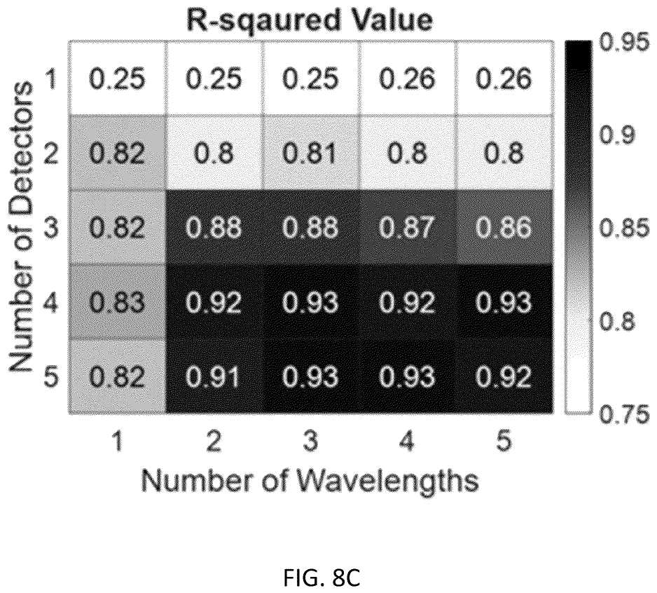

FIGS. 8(a), 8(b), and 8(c) show the results for the high melanin test population when the calibration equation was generated using the representative population. More specifically, FIG. 8(a) shows that the bias is reduced with an increased number of wavelengths and detectors. FIG. 8(b) shows that the spread of LOA is reduced with an increased number of wavelengths and detectors, but the impact of the increase in the number of detectors is greater.

FIG. 8(c) shows that r² values also are increased with higher combinations of detectors and wavelengths.

FIGS. 8(a), 8(b), and 8(c) further show that when one detector and one wavelength are used, the bias and LOA spread are higher and the r² value is lower. When only one detector is used, with an increased number of wavelengths the bias and LOA spread are not reduced highly. But, with an increased number of detectors the impact is much higher. The bias and spread of LOA are reduced with an increased number of detectors along with an increased number of wavelengths. For a given number of wavelengths, increasing the number of detectors improves the performance.

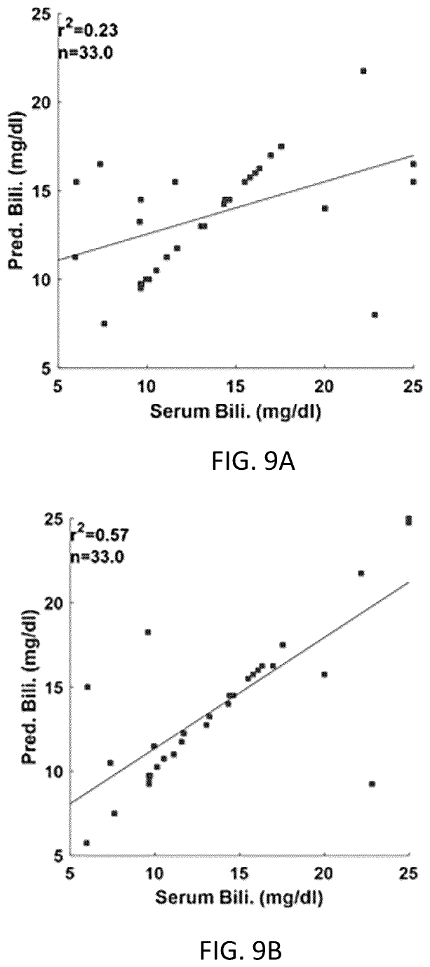

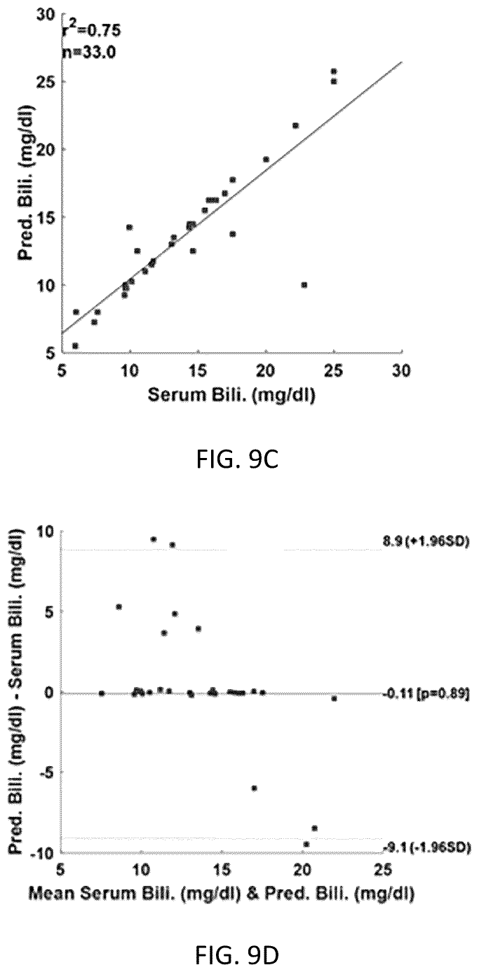

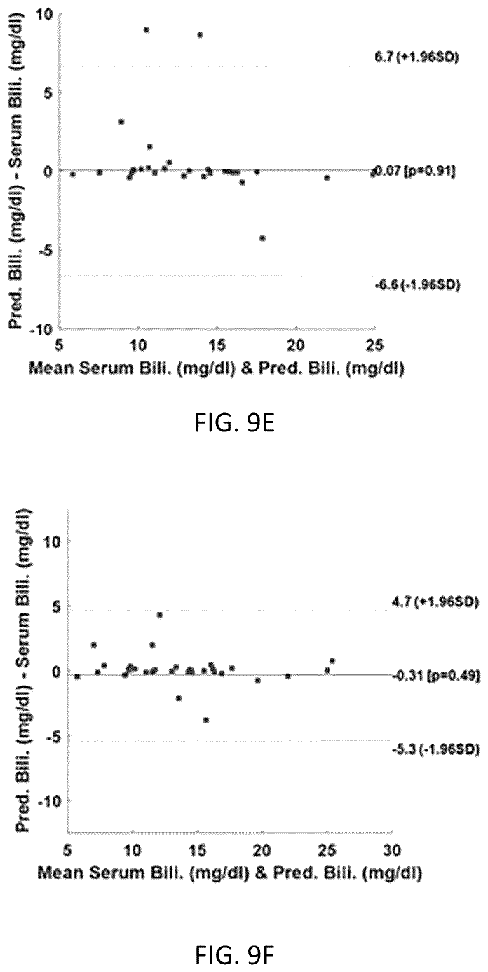

FIG. 9(a) shows the r² value and FIG. 9(d) shows the spread of LOA when one ROI (similar to one detector) is used in each of the RGB channels. The r2 value is lower when only one detector is used. Also, the spread of LOA is high (±9 mg/dl).

FIG. 9(b) shows the r² value and FIG. 9(e) shows the spread of LOA when two ROI are used in each of the RGB channels. The r² value increased to 0.57 from 0.23 with two ROI as opposed to one ROI. The spread of LOA is reduced when more ROI are used in each channel. Specifically, compare FIG. 9(e) (±6 mg/dl). to FIG. 93(d) (±9 mg/dl).

Finally, FIG. 9(c) shows the r² value and FIG. 9(f) shows the spread of LOA when three ROI are used in each of the RGB channels. The r2 value increased when compared to one or two ROI. The spread of LOA is reduced from (±9 mg/dl) to (±6 mg/dl) to (±5 mg/dl) when one or two or and three ROI are used, respectively.

It is evident that with an increased number of detectors per color channel, the accuracy of bilirubin prediction increases which is similar to the MC simulation results.

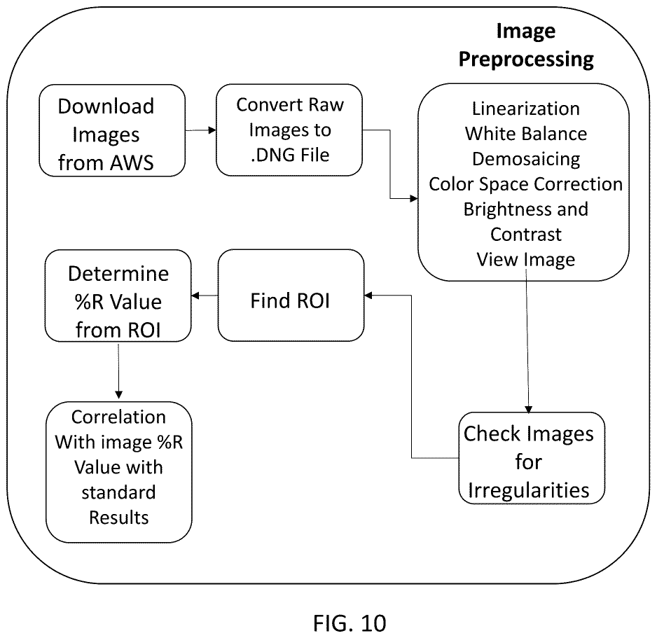

TcB devices are essential diagnostic devices which are used widely to detect bilirubin in newborn infants. It is important that TcB devices accurately estimate bilirubin levels in order to avoid either under-treatment or over-utilization of treatment resources. TcB measurements have shown reduced accuracy in highly pigmented infants. This disclosure uses MC simulations to investigate the basis of quantification error, and additionally describes an approach for addressing racial bias in TcB measurements. These advantages are explored through the evaluation of MC-simulated neonatal skin with the disclosed mobile phone TcB system. Linear regression models were created from the simulated images with varying combinations of ROI and wavelengths. Validation of these model creations were conducted from study data collected with the disclosed mobile phone TcB system. Both MC simulation and mobile phone TcB image analysis (FIG. 10 is a flow chart summarizing the image analysis) showed an improvement in linear regression statistics and LOA as the ROI and wavelengths of the model were increased. These results suggest increasing model parameterization through ROI with increased source detector offsets and multiple wavelengths.

One approach to addressing the problems with transcutaneous optical measurements is based on computer simulations of light propagation through tissue. Although it is understood that reflectance measured at tissue surface locations with different spatial offsets from a single-position light source incident on the skin travel different mean-free-paths through the tissue, computer simulations also indicate that these different locations have unique relative ratios of contributions from different tissue layers. In order to realize a multiple of different spatial offset measurement positions, the disclosed mobile phone TcB system collects a 2D map of the entire reflectance spatial decay profile, in three different detector color channels, with potentially many different wavelength light sources. Small regions in these reflectance spatial decay maps can then be selected as the elements in a multivariate regression model to approximate the primary chromophore of interest. As a result, the disclosed mobile phone TcB system contains a far higher dimensionality of spatial channels in comparison with conventional bilirubinometers which only have one or two spatial offset paths. In addition, the disclosed mobile phone TcB system uses optimization algorithms to determine the preferred spatial offset schema with which to construct models for specific devices. As a result, computer simulations suggest that in the specific case of bias and error associated with race and ethnicity, the disclosed mobile phone TcB system has less bias and error than current techniques. Interest in transcutaneous measurements of physiological parameters has exploded recently with the prevalence of wearable consumer electronics, and the disclosed mobile phone TcB system is well suited for integration in wearables because the system relies on commoditized camera sensor modules available at production-scale costs.

The studies described above included computer simulations of the technique for estimation of both oxygenation saturation levels and bilirubin levels, indicating reduced pigmentation-dependent measurement bias and error across both scenarios when regression models are created using multiple regions of interest digitally defined across the 2D sensor field of view. In addition, studies using the disclosed mobile phone camera platform were completed to quantify bilirubin in neonates. The results indicate a good ability to predict bilirubin.

The disclosure includes an optical detection system in which a 2D sensor (camera) images the diffuse reflectance spatial decay function generated from a light source or multiple light sources incident on the surface of the tissue at a certain distance offset from the camera's field of view. A multi-variate regression model is then constructed against the gold standard clinical measurement (i.e., TSB in bilirubinometry) using multiple spatial regions of interest in the camera field of view. In a color camera, there are three different (red/green/blue) detector color channels from which to extract unique regions of interest, as is the case in bilirubinometry. An optimization algorithm is then used to identify the coordinates and sizes for regions of interest which minimize model error. The system can comprise a snap-on adapter to any consumer camera or mobile phone camera, or alternatively be a custom built device centered around a consumer 2D imaging module.

Computer simulations were performed for the disclosed mobile phone system in bilirubinometry. Disclosed is a mobile phone camera-based device that was used to perform human subject tests on infants across three sites. Optimization algorithms can be generated that inform selection of the ROI which are used to create the regression model. The approach is fundamentally a regression-based approach, and thus requires construction of a model against gold standard-blood based measurements.

Various embodiments of end products incorporating the disclosed system are contemplated. One end product is a point-of-care bilirubinometer for screening newborns for neonatal jaundice, either as a smart phone case style adaptor/mobile phone app pair for at-home use by nurse midwives or parents, or a specific-use handheld measurement device for use in low-resource settings globally.

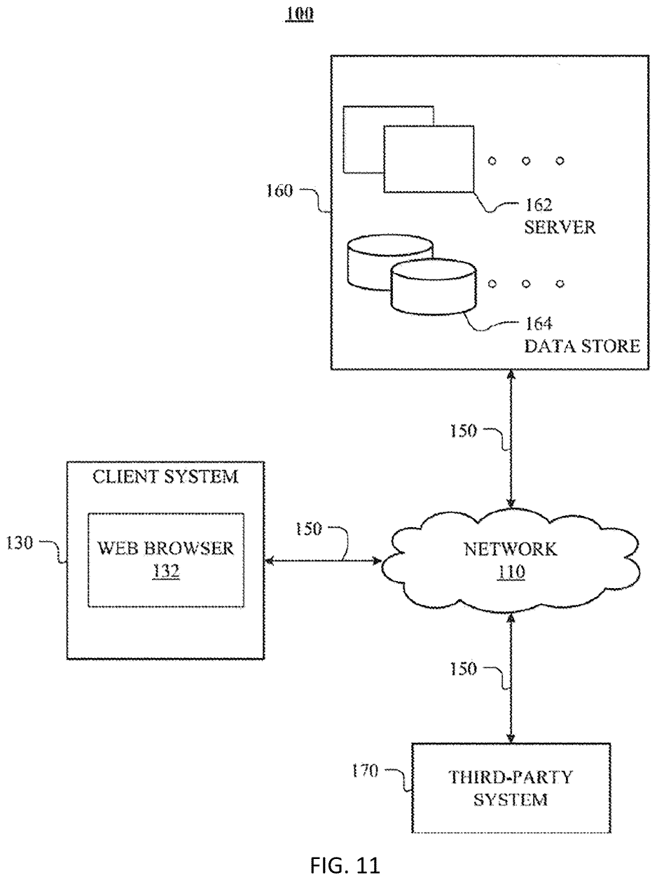

FIG. 11 illustrates an example network environment 100 associated with the disclosed mobile phone TcB system 160. The network environment 100 includes a client system 130, the disclosed mobile phone TcB system 160, and a third-party system 170 connected to each other by a network 110. Although FIG. 11 illustrates a particular arrangement of the client system 130, the disclosed mobile phone TcB system 160, the third-party system 170, and the network 110, this disclosure contemplates any suitable arrangement of the client system 130, the disclosed mobile phone TcB system 160, the third-party system 170, and the network 110. As an example and not by way of limitation, two or more of the client system 130, the disclosed mobile phone TcB system 160, and the third-party system 170 may be connected to each other directly, bypassing the network 110. As another example, two or more of the client system 130, the disclosed mobile phone TcB system 160, and the third-party system 170 may be physically or logically co-located with each other in whole or in part. Moreover, although FIG. 11 illustrates a particular number of client systems 130, disclosed mobile phone TcB systems 160, third-party systems 170, and networks 110, this disclosure contemplates any suitable number of client systems 130, disclosed mobile phone TcB systems 160, third-party systems 170, and networks 110. As an example and not by way of limitation, the network environment 100 may include multiple client systems 130, disclosed mobile phone TcB systems 160, third-party systems 170, and networks 110.

This disclosure contemplates any suitable network 110. As an example and not by way of limitation, one or more portions of the network 110 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. The network 110 may include one or more networks 110.

One or more links 150 may connect the client system 130, the disclosed mobile phone TcB system 160, and the third-party system 170 to the communication network 110 or to each other. This disclosure contemplates any suitable links 150. In particular embodiments, the one or more links 150 include one or more wireline (such as, for example, Digital Subscriber Line (DSL) or Data Over Cable Service Interface Specification (DOC SIS)), wireless (such as, for example, Wi-Fi or Worldwide Interoperability for Microwave Access (WiMAX)), or optical (such as, for example, Synchronous Optical Network (SONET) or Synchronous Digital Hierarchy (SDH)) links. In particular embodiments, the one or more links 150 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 150, or a combination of two or more such links 150. The links 150 need not necessarily be the same throughout the network environment 100. The one or more first links 150 may differ in one or more respects from one or more second links 150.

In particular embodiments, the client system 130 may be an electronic device including hardware, software, or embedded logic components or a combination of two or more such components and capable of carrying out the appropriate functionalities implemented or supported by the client system 130. As an example and not by way of limitation, the client system 130 may include a computer system such as a desktop computer, notebook or laptop computer, netbook, a tablet computer, e-book reader, GPS device, camera, personal digital assistant (PDA), handheld electronic device, cellular telephone, smartphone, other suitable electronic device, or any suitable combination thereof. This disclosure contemplates any suitable client systems 130. The client system 130 may enable a network user at the client system 130 to access the network 110. The client system 130 may enable its user to communicate with other users at other client systems 130.

In particular embodiments, the client system 130 may include a web browser 132, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user of the client system 130 may enter a Uniform Resource Locator (URL) or other address directing the web browser 132 to a particular server (such as a server 162, or a server associated with a third-party system 170), and the web browser 132 may generate a Hyper Text Transfer Protocol (HTTP) request and communicate the HTTP request to the server. The server may accept the HTTP request and communicate to the client system 130 one or more Hyper Text Markup Language (HTML) files responsive to the HTTP request. The client system 130 may render a webpage based on the HTML files from the server for presentation to the user. This disclosure contemplates any suitable webpage files. As an example and not by way of limitation, webpages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. In this document, reference to a webpage encompasses one or more corresponding webpage files (which a browser may use to render the webpage) and vice versa, where appropriate.

In particular embodiments, the disclosed mobile phone TcB system 160 may include a network-addressable computing system that can host an online analytical engine. The disclosed mobile phone TcB system 160 may generate, store, receive, and send data related to the system, subject to laws and regulations regarding that data. The disclosed mobile phone TcB system 160 may be accessed by the other components of the network environment 100 either directly or via the network 110. In particular embodiments, the disclosed mobile phone TcB system 160 may receive inputs from one or more of a performance engine or an experience engine (which may be independent systems or sub-systems of the disclosed mobile phone TcB system 160). The performance engine may receive data. The experience engine may receive data. In particular embodiments, the disclosed mobile phone TcB system 160 may include one or more servers 162. Each server 162 may be a unitary server or a distributed server spanning multiple computers or multiple datacenters. The servers 162 may be of various types, such as, for example and without limitation, web server, news server, mail server, message server, advertising server, file server, application server, exchange server, database server, proxy server, another server suitable for performing functions or processes described in this document, or any combination thereof. In particular embodiments, each server 162 may include hardware, software, or embedded logic components or a combination of two or more such components for carrying out the appropriate functionalities implemented or supported by the server 162. In particular embodiments, the disclosed mobile phone TcB system 160 may include one or more data stores 164. The data stores 164 may be used to store various types of information. In particular embodiments, the information stored in the data stores 164 may be organized according to specific data structures. In particular embodiments, each data store 164 may be a relational, columnar, correlation, or another suitable database. Although this disclosure describes or illustrates particular types of databases, this disclosure contemplates any suitable types of databases. Particular embodiments may provide interfaces that enable the client system 130, the disclosed mobile phone TcB system 160, or the third-party system 170 to manage, retrieve, modify, add, or delete, the information stored in the data store 164.

In particular embodiments, the disclosed mobile phone TcB system 160 may be capable of linking a variety of entities. As an example and not by way of limitation, the disclosed mobile phone TcB system 160 may enable users to interact with each other as well as receive content from the third-party systems 170 or other entities, or allow users to interact with these entities through an application programming interface (API) or other communication channels.

In particular embodiments, the third-party system 170 may include one or more types of servers, one or more data stores, one or more interfaces, including but not limited to APIs, one or more web services, one or more content sources, one or more networks, or any other suitable components, e.g., with which servers may communicate. The third-party system 170 may be operated by a different entity from an entity operating the disclosed mobile phone TcB system 160. In particular embodiments, however, the disclosed mobile phone TcB system 160 and the third-party systems 170 may operate in conjunction with each other to provide services to users of the disclosed mobile phone TcB system 160 or the third-party systems 170. In this sense, the disclosed mobile phone TcB system 160 may provide a platform, or backbone, which other systems, such as the third-party systems 170, may use to provide services and functionality to users across the Internet.

In particular embodiments, the disclosed mobile phone TcB system 160 also includes user-generated content objects, which may enhance the interactions of a user with the disclosed mobile phone TcB system 160. User-generated content may include anything a user can add, upload, send, or “post” to the disclosed mobile phone TcB system 160. In particular embodiments, user-generated content may comprise user-profile information. As an example and not by way of limitation, a user communicates posts to the disclosed mobile phone TcB system 160 from the client system 130. Posts may include data such as neonatal records, other textual data, location information, graphs, videos, links, or other similar data or content. Content may also be added to the disclosed mobile phone TcB system 160 by a third-party through a suitable communication channel.

In particular embodiments, the disclosed mobile phone TcB system 160 may include a variety of servers, sub-systems, programs, modules, logs, and data stores. In particular embodiments, the disclosed mobile phone TcB system 160 may include one or more of the following: a web server, action logger, API-request server, relevance-and-ranking engine, content-object classifier, notification controller, action log, third-party-content-object-exposure log, inference module, authorization/privacy server, search module, advertisement-targeting module, user-interface module, user/patient-profile store, connection store, third-party content store, or location store. The disclosed mobile phone TcB system 160 may also include suitable components such as network interfaces, security mechanisms, load balancers, failover servers, management-and-network-operations consoles, other suitable components, or any suitable combination thereof. In particular embodiments, the disclosed mobile phone TcB system 160 may include one or more user-profile stores for storing user profiles. A user/researcher profile may include, for example, neonatal information, biographic information, demographic information, behavioral information, social information, medical information, or other types of descriptive information, such as experience, history, preferences, or location. A web server may be used to link the disclosed mobile phone TcB system 160 to one or more client systems 130 or one or more third-party systems 170 via the network 110. The web server may include a mail server or other messaging functionality for receiving and routing messages between the disclosed mobile phone TcB system 160 and one or more of the client systems 130. An API-request server may allow the third-party system 170 to access information from the disclosed mobile phone TcB system 160 by calling one or more APIs. An action logger may be used to receive communications from a web server about the actions of a user on or off the disclosed mobile phone TcB system 160. In conjunction with the action log, a third-party-content-object log may be maintained of user exposures to third-party-content objects. A notification controller may provide information regarding content objects to the client system 130. Information may be pushed to the client system 130 as notifications, or information may be pulled from the client system 130 responsive to a request received from the client system 130. Authorization servers may be used to enforce one or more privacy settings of the users of the disclosed mobile phone TcB system 160. A privacy setting of a user determines how particular information associated with a user can be shared. The authorization server may allow users to opt in to or opt out of having their actions logged by the disclosed mobile phone TcB system 160 or shared with other systems (e.g., the third-party system 170), such as, for example, by setting appropriate privacy settings. Third-party-content-object stores may be used to store content objects received from third parties, such as the third-party system 170. Location stores may be used to store location information received from the client system 130 associated with users.

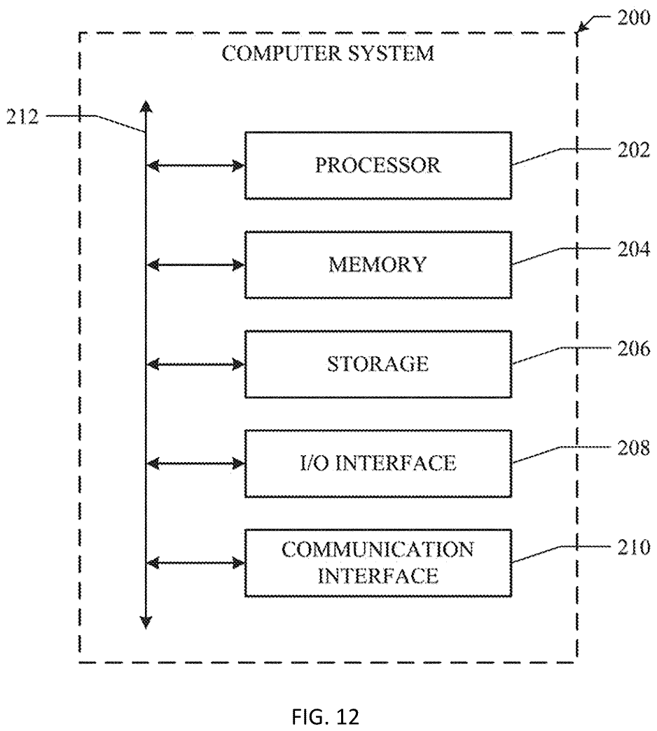

FIG. 12 illustrates an example computer system 200. In particular embodiments, one or more computer systems 200 perform one or more steps of one or more methods described or illustrated in this document. In particular embodiments, one or more computer systems 200 provide functionality described or illustrated in this document. In particular embodiments, software running on one or more computer systems 200 performs one or more steps of one or more methods described or illustrated in this document or provides functionality described or illustrated in this document. Particular embodiments include one or more portions of one or more computer systems 200. In this document, reference to a computer system may encompass a computing device, and vice versa, where appropriate. Moreover, reference to a computer system may encompass one or more computer systems, where appropriate.

This disclosure contemplates any suitable number of computer systems 200. This disclosure contemplates the computer system 200 taking any suitable physical form. As example and not by way of limitation, the computer system 200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these devices. Where appropriate, the computer system 200 may include one or more computer systems 200; be unitary or distributed; span multiple locations; span multiple machines; span multiple data centers; or reside in a cloud, which may include one or more cloud components in one or more networks. Where appropriate, one or more computer systems 200 may perform without substantial spatial or temporal limitation one or more steps of one or more methods described or illustrated in this document. As an example and not by way of limitation, the one or more computer systems 200 may perform in real time or in batch mode one or more steps of one or more methods described or illustrated in this document. The one or more computer systems 200 may perform at different times or at different locations one or more steps of one or more methods described or illustrated in this document, where appropriate.

In particular embodiments, the computer system 200 includes a processor 202, memory 204, storage 206, an input/output (I/O) interface 208, a communication interface 210, and a bus 212. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 202 includes hardware for executing instructions, such as those making up a computer program. As an example and not by way of limitation, to execute instructions, the processor 202 may retrieve (or fetch) the instructions from an internal register, an internal cache, the memory 204, or the storage 206; decode and execute them; and then write one or more results to an internal register, an internal cache, the memory 204, or the storage 206. In particular embodiments, the processor 202 may include one or more internal caches for data, instructions, or addresses. This disclosure contemplates the processor 202 including any suitable number of any suitable internal caches, where appropriate. As an example and not by way of limitation, the processor 202 may include one or more instruction caches, one or more data caches, and one or more translation lookaside buffers (TLBs). Instructions in the instruction caches may be copies of instructions in the memory 204 or the storage 206, and the instruction caches may speed up retrieval of those instructions by the processor 202. Data in the data caches may be copies of data in the memory 204 or the storage 206 for instructions executing at the processor 202 to operate on; the results of previous instructions executed at the processor 202 for access by subsequent instructions executing at the processor 202 or for writing to the memory 204 or the storage 206; or other suitable data. The data caches may speed up read or write operations by the processor 202. The TLBs may speed up virtual-address translation for the processor 202. In particular embodiments, the processor 202 may include one or more internal registers for data, instructions, or addresses. This disclosure contemplates the processor 202 including any suitable number of any suitable internal registers, where appropriate. Where appropriate, the processor 202 may include one or more arithmetic logic units (ALUs); be a multi-core processor; or include one or more processors 202. Although this disclosure describes and illustrates a particular processor, this disclosure contemplates any suitable processor.

In particular embodiments, the memory 204 includes main memory for storing instructions for the processor 202 to execute or data for the processor 202 to operate on. As an example and not by way of limitation, the computer system 200 may load instructions from the storage 206 or another source (such as, for example, another computer system 200) to the memory 204. The processor 202 may then load the instructions from the memory 204 to an internal register or internal cache. To execute the instructions, the processor 202 may retrieve the instructions from the internal register or internal cache and decode them. During or after execution of the instructions, the processor 202 may write one or more results (which may be intermediate or final results) to the internal register or internal cache. The processor 202 may then write one or more of those results to the memory 204. In particular embodiments, the processor 202 executes only instructions in one or more internal registers or internal caches or in the memory 204 (as opposed to the storage 206 or elsewhere) and operates only on data in one or more internal registers or internal caches or in the memory 204 (as opposed to the storage 206 or elsewhere). One or more memory buses (which may each include an address bus and a data bus) may couple the processor 202 to the memory 204. The bus 212 may include one or more memory buses, as described below. In particular embodiments, one or more memory management units (MMUs) reside between the processor 202 and the memory 204 and facilitate accesses to the memory 204 requested by the processor 202. In particular embodiments, the memory 204 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. The memory 204 may include one or more memories 204, where appropriate. Although this disclosure describes and illustrates particular memory, this disclosure contemplates any suitable memory.

In particular embodiments, the storage 206 includes mass storage for data or instructions. As an example and not by way of limitation, the storage 206 may include a hard disk drive (HDD), a floppy disk drive, flash memory, an optical disc, a magneto-optical disc, magnetic tape, or a Universal Serial Bus (USB) drive or a combination of two or more of these. The storage 206 may include removable or non-removable (or fixed) media, where appropriate. The storage 206 may be internal or external to the computer system 200, where appropriate. In particular embodiments, the storage 206 is non-volatile, solid-state memory. In particular embodiments, the storage 206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates the storage 206 taking any suitable physical form. The storage 206 may include one or more storage control units facilitating communication between the processor 202 and the storage 206, where appropriate. Where appropriate, the storage 206 may include one or more storages 206. Although this disclosure describes and illustrates particular storage, this disclosure contemplates any suitable storage.

In particular embodiments, the I/O interface 208 includes hardware, software, or both, providing one or more interfaces for communication between the computer system 200 and one or more I/O devices. The computer system 200 may include one or more of these I/O devices, where appropriate. One or more of these I/O devices may enable communication between a person and the computer system 200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 208 for them. Where appropriate, the I/O interface 208 may include one or more device or software drivers enabling the processor 202 to drive one or more of these I/O devices. The I/O interface 208 may include one or more I/O interfaces 208, where appropriate. Although this disclosure describes and illustrates a particular I/O interface, this disclosure contemplates any suitable I/O interface.

In particular embodiments, the communication interface 210 includes hardware, software, or both providing one or more interfaces for communication (such as, for example, packet-based communication) between the computer system 200 and one or more other computer systems 200 or one or more networks. As an example and not by way of limitation, the communication interface 210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 210 for it. As an example and not by way of limitation, the computer system 200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computer system 200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. The computer system 200 may include any suitable communication interface 210 for any of these networks, where appropriate. The communication interface 210 may include one or more communication interfaces 210, where appropriate. Although this disclosure describes and illustrates a particular communication interface, this disclosure contemplates any suitable communication interface.

In particular embodiments, the bus 212 includes hardware, software, or both coupling components of the computer system 200 to each other. As an example and not by way of limitation, the bus 212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. The bus 212 may include one or more buses 212, where appropriate. Although this disclosure describes and illustrates a particular bus, this disclosure contemplates any suitable bus or interconnect.

In this document, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

This disclosure contemplates one or more computer-readable storage media implementing any suitable storage. In particular embodiments, a computer-readable storage medium implements one or more portions of the processor 202 (such as, for example, one or more internal registers or caches), one or more portions of the memory 204, one or more portions of the storage 206, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody software. In this document, reference to software may encompass one or more applications, bytecode, one or more computer programs, one or more executables, one or more instructions, logic, machine code, one or more scripts, or source code, and vice versa, where appropriate. In particular embodiments, software includes one or more application programming interfaces (APIs). This disclosure contemplates any suitable software written or otherwise expressed in any suitable programming language or combination of programming languages. In particular embodiments, software is expressed as source code or object code. In particular embodiments, software is expressed in a higher-level programming language, such as, for example, C, Perl, or a suitable extension thereof. In particular embodiments, software is expressed in a lower-level programming language, such as assembly language (or machine code). In particular embodiments, software is expressed in JAVA. In particular embodiments, software is expressed in Hyper Text Markup Language (HTML), Extensible Markup Language (XML), JavaScript Object Notation (JSON) or other suitable markup language.

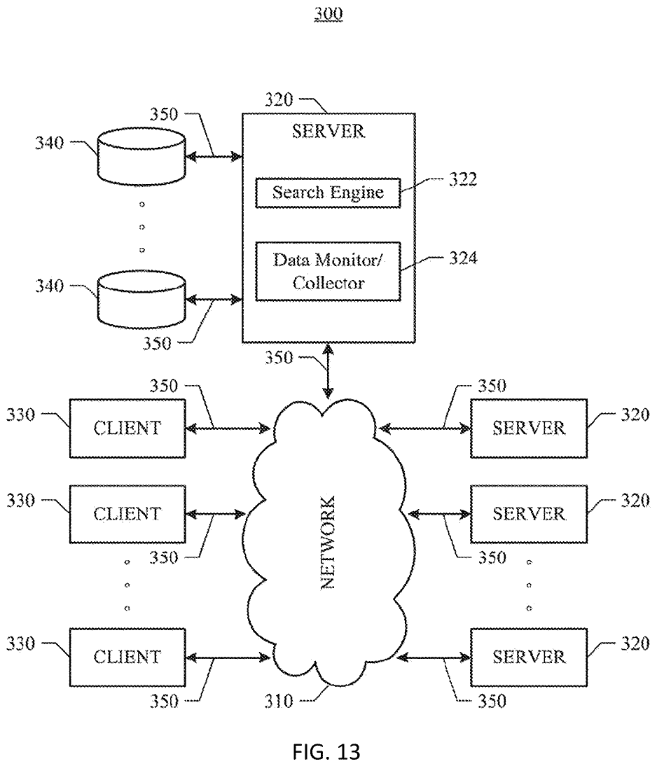

FIG. 13 illustrates an example network environment 300. This disclosure contemplates any suitable network environment 300. As an example and not by way of limitation, although this disclosure describes and illustrates the network environment 300 as implementing a client-server model, this disclosure contemplates one or more portions of the network environment 300 being peer-to-peer, where appropriate. Particular embodiments may operate in whole or in part in one or more network environments 300. In particular embodiments, one or more elements of the network environment 300 provide functionality described or illustrated in this document. Particular embodiments include one or more portions of the network environment 300. The network environment 300 includes a network 310 coupling one or more servers 320 and one or more clients 330 to each other. This disclosure contemplates any suitable network 310. As an example and not by way of limitation, one or more portions of the network 310 may include an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the Public Switched Telephone Network (PSTN), a cellular telephone network, or a combination of two or more of these. The network 310 may include one or more networks 310.

One or more links 350 couple the servers 320 and the clients 330 to the network 310 or to each other. This disclosure contemplates any suitable links 350. As an example and not by way of limitation, the one or more links 350 each include one or more wireline (such as, for example, Digital Subscriber Line (DSL) or Data Over Cable Service Interface Specification (DOCSIS)), wireless (such as, for example, Wi-Fi or Worldwide Interoperability for Microwave Access (WiMAX)) or optical (such as, for example, Synchronous Optical Network (SONET) or Synchronous Digital Hierarchy (SDH)) links 350. In particular embodiments, the one or more links 350 each includes an intranet, an extranet, a VPN, a LAN, a WLAN, a WAN, a MAN, a communications network, a satellite network, a portion of the Internet, or another link 350 or a combination of two or more such links 350. The links 350 need not necessarily be the same throughout the network environment 300. One or more first links 350 may differ in one or more respects from one or more second links 350.

This disclosure contemplates any suitable servers 320. As an example and not by way of limitation, one or more servers 320 may each include one or more advertising servers, applications servers, catalog servers, communications servers, database servers, exchange servers, fax servers, file servers, game servers, home servers, mail servers, message servers, news servers, name or DNS servers, print servers, proxy servers, sound servers, standalone servers, web servers, or web-feed servers. In particular embodiments, the server 320 includes hardware, software, or both for providing the functionality of the server 320. As an example and not by way of limitation, the server 320 operates as a web server and may be capable of hosting websites containing web pages or elements of web pages and includes appropriate hardware, software, or both for doing so. In particular embodiments, a web server may host HTML or other suitable files or dynamically create or constitute files for web pages on request. In response to a Hyper Text Transfer Protocol (HTTP) or other request from the client 330, the web server may communicate one or more such files to the client 330. As another example, the server 320 that operates as a mail server may be capable of providing e-mail services to one or more clients 330. As another example, the server 320 that operates as a database server may be capable of providing an interface for interacting with one or more data stores (such as, for example, a data store 340 described below). Where appropriate, the server 320 may include one or more servers 320; be unitary or distributed; span multiple locations; span multiple machines; span multiple datacenters; or reside in a cloud, which may include one or more cloud components in one or more networks.

In particular embodiments, the one or more links 350 may couple the server 320 to one or more data stores 340. The data store 340 may store any suitable information, and the contents of the data store 340 may be organized in any suitable manner. As an example and not by way or limitation, the contents of the data store 340 may be stored as a dimensional, flat, hierarchical, network, object-oriented, relational, XML, NoSQL, Hadoop, or other suitable database or a combination or two or more of these. The data store 340 (or the server 320 coupled to it) may include a database-management system or other hardware or software for managing the contents of the data store 340. The database-management system may perform read and write operations, delete or erase data, perform data deduplication, query or search the contents of the data store 340, or provide other access to the data store 340.

In particular embodiments, the one or more servers 320 may each include one or more search engines 322. The search engine 322 may include hardware, software, or both for providing the functionality of the search engine 322. As an example and not by way of limitation, the search engine 322 may implement one or more search algorithms to identify network resources in response to search queries received at the search engine 322, one or more ranking algorithms to rank identified network resources, or one or more summarization algorithms to summarize identified network resources. In particular embodiments, a ranking algorithm implemented by the search engine 322 may use a machine-learned ranking formula, which the ranking algorithm may obtain automatically from a set of training data constructed from pairs of search queries and selected Uniform Resource Locators (URLs), where appropriate.

In particular embodiments, the one or more servers 320 may each include one or more data monitors/collectors 324. The data monitor/collection 324 may include hardware, software, or both for providing the functionality of the data collector/collector 324. As an example and not by way of limitation, the data monitor/collector 324 at the server 320 may monitor and collect network-traffic data at the server 320 and store the network-traffic data in the one or more data stores 340. In particular embodiments, the server 320 or another device may extract pairs of search queries and selected URLs from the network-traffic data, where appropriate.

This disclosure contemplates any suitable clients 330. The client 330 may enable a user at the client 330 to access or otherwise communicate with the network 310, the servers 320, or other clients 330. As an example and not by way of limitation, the client 330 may have a web browser, such as MICROSOFT INTERNET EXPLORER or MOZILLA FIREFOX, and may have one or more add-ons, plug-ins, or other extensions, such as GOOGLE TOOLBAR or YAHOO TOOLBAR. The client 330 may be an electronic device including hardware, software, or both for providing the functionality of the client 330. As an example and not by way of limitation, the client 330 may, where appropriate, be an embedded computer system, an SOC, an SBC (such as, for example, a COM or SOM), a desktop computer system, a laptop or notebook computer system, an interactive kiosk, a mainframe, a mesh of computer systems, a mobile telephone, a PDA, a netbook computer system, a server, a tablet computer system, or a combination of two or more of these. Where appropriate, the client 330 may include one or more clients 330; be unitary or distributed; span multiple locations; span multiple machines; span multiple datacenters; or reside in a cloud, which may include one or more cloud components in one or more networks.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

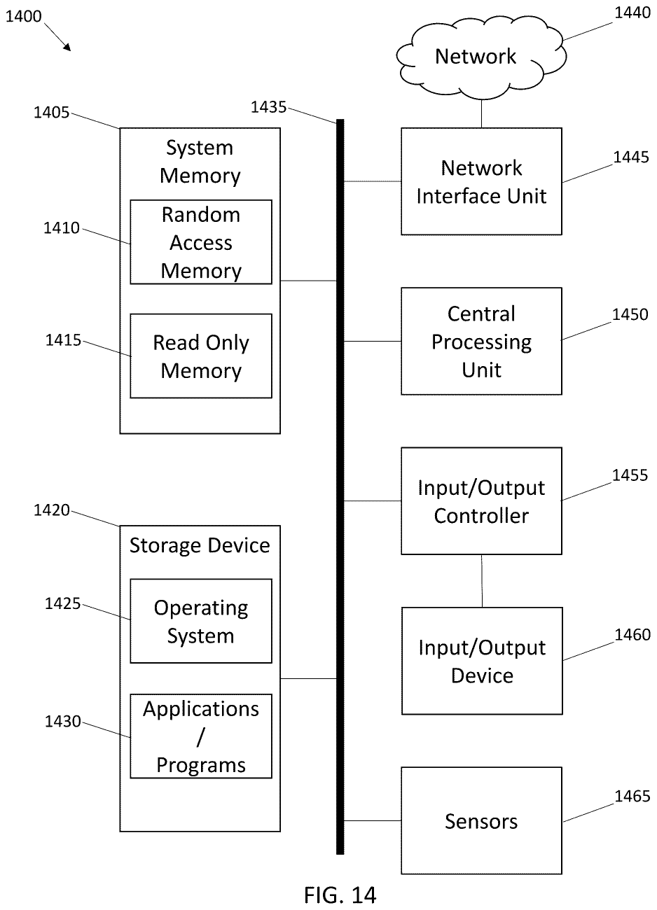

FIG. 14 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 14 depicts an illustrative computer architecture for a computer 1400 for practicing the various embodiments of the invention. The computer architecture shown in FIG. 14 illustrates a conventional personal computer, including a central processing unit 1450 (“CPU”), a system memory 1405, including a random-access memory 1410 (“RAM”) and a read-only memory (“ROM”) 1415, and a system bus 1435 that couples the system memory 1405 to the CPU 1450. A basic input/output system containing the basic routines that help to transfer information between elements within the computer, such as during startup, is stored in the ROM 1415. The computer 1400 further includes a storage device 1420 for storing an operating system 1425, application/program 1430, and data.

The storage device 1420 is connected to the CPU 1450 through a storage controller (not shown) connected to the bus 1435. The storage device 1420 and its associated computer-readable media, provide non-volatile storage for the computer 1400. Although the description of computer-readable media contained herein refers to a storage device, such as a hard disk or CD-ROM drive, it should be appreciated by those skilled in the art that computer-readable media can be any available media that can be accessed by the computer 1400.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1400 may operate in a networked environment using logical connections to remote computers through a network 1440, such as TCP/IP network such as the Internet or an intranet. The computer 1400 may connect to the network 1440 through a network interface unit 1445 connected to the bus 1435. It should be appreciated that the network interface unit 1445 may also be utilized to connect to other types of networks and remote computer systems.

The computer 1400 may also include an input/output controller 1455 for receiving and processing input from a number of input/output devices 1460, including a keyboard, a mouse, a touchscreen, a camera, a microphone, a controller, a joystick, or other type of input device. Similarly, the input/output controller 1455 may provide output to a display screen, a printer, a speaker, or other type of output device. The computer 1400 can connect to the input/output device 1460 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1420 and RAM 1410 of the computer 1400, including an operating system 1425 suitable for controlling the operation of a networked computer. The storage device 1420 and RAM 1410 may also store one or more applications/programs 1430. In particular, the storage device 1420 and RAM 1410 may store an application/program 1430 for providing a variety of functionalities to a user. For instance, the application/program 1430 may comprise many types of programs such as a word processing application, a spreadsheet application, a desktop publishing application, a database application, a gaming application, internet browsing application, electronic mail application, messaging application, and the like. According to an embodiment of the present invention, the application/program 1430 comprises a multiple functionality software application for providing word processing functionality, slide presentation functionality, spreadsheet functionality, database functionality and the like.

The computer 1400 in some embodiments can include a variety of sensors 1465 for monitoring the environment surrounding and the environment internal to the computer 1400. These sensors 1465 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Although illustrated and described above with reference to certain specific embodiments and examples, the present disclosure is nevertheless not intended to be limited to the details shown. Rather, various modifications may be made in the details within the scope and range of equivalents of the claims and without departing from the spirit of the disclosure.

The following publications are each hereby incorporated herein by reference in their entirety:
Harrison-Smith B, Dumont A P, Arefin M S, Sun Y, Lawal N, Dobson D, Nwaba A, Grossarth S, Paed A M, Farouk Z L, Weitkamp J H, Patil C A. Development of a mobile phone camera-based transcutaneous bilirubinometer for low-resource settings. Biomed Opt Express. 2022 Apr. 15; 13(5):2797-2809. doi: 10.1364/BOE.449625. PMID: 35774304; PMCID: PMC 9203089.
Alexander P. Dumont, Brandon Harrison, Zachary T. McCormick, Nishant Ganesh Kumar, Chetan A. Patil, “Development of mobile phone based transcutaneous billirubinometry,” Proc.

SPIE 10055, Optics and Biophotonics in Low-Resource Settings III, 100550T (3 Mar. 2017); https://doi.org/10.1117/12.2257428

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed:

1. A transcutaneous spatially resolved diffuse reflectance optical measurement system, comprising:
a photodetector or multi-channel 2D sensor array;
at least one light source; and
a computing system communicatively connected to the photodetector and the light source;
wherein the system is configured to perform wavelength-resolved measurements.

2. The system of claim 1, further comprising a filter housing including at least one filter, wherein the filter housing is configured to be positioned between the light source, the photodetector and a skin sample to be measured, wherein the light source and the photodetector are positioned on a first side of the filter housing, and the skin sample to be measured is positioned on a second side of the filter housing opposite the first side.

3. The system of claim 1, wherein the computing system comprises a processor and a non-transitory computer-readable medium with instructions stored thereon, which when executed by the processor, perform steps comprising:
calibrating the system;
measuring a skin sample via transcutaneous spatially resolved diffuse reflectance;
calculating a bilirubin concentration withing the skin sample; and
displaying the bilirubin concentration.

4. The system of claim 1, wherein the light source comprises a mobile phone flash LED, and the photodetector comprises a mobile phone camera.

5. The system of claim 2, wherein the filter housing further comprises an adaptor or a case suitable to connect to a mobile phone.

6. The system of claim 5, wherein the filter housing is configured to align the at least one filter with the light source or photodetector.

7. The system of claim 2, wherein the at least one filter comprises first and second triple-band pass filters.

8. The system of claim 7, wherein the first triple-band pass filter is positioned proximate to the light source, and the second triple-band pass filter is positioned proximate to the photodetector.

9. The system of claim 7, wherein the first and second triple-band pass filters have identical spectral profiles.

10. The system of claim 1, wherein the light source is offset from a field of view of the photo detector in a range of 0 mm to 100 mm.

11. The system of claim 1, wherein the system is configured to measure in a spectral range of 300 nm to 2000 nm.

12. The system of claim 2, wherein the filter housing further comprises an angled channel configured to direct light from the light source to the field of view of the photodetector.

13. The system of claim 12, wherein light from the light source is filtered and enters the skin sample at a 15 degree to 45 degree angle with a 0 mm to 20 mm source-to-detector separation distance.

14. The system of claim 12, wherein light from the light source is filtered and enters the skin sample at a 30 degree angle with a 0 mm source-to-detector separation distance.

15. The system of claim 2, wherein the filter housing comprises a highly opaque material configured to minimize light transmission through the material.

16. The system of claim 2, wherein the filter housing further comprises a pinhole of diameter 0.1 mm to 5 mm configured to reduce a half angle of the light source.

17. A transcutaneous spatially resolved diffuse reflectance optical measurement method to measure bilirubin concentration, comprising:
providing the transcutaneous spatially resolved diffuse reflectance optical measurement system of claim 1;
calibrating the system;
measuring a skin sample via transcutaneous spatially resolved diffuse reflectance;
calculating a bilirubin concentration withing the skin sample; and
displaying the bilirubin concentration.

18. The method of claim 17, wherein the step of calibrating the system comprises measuring against one or more white or gray samples using a measurement sequence comprising obtaining a plurality reflectance images with the light source on, and obtaining a plurality of background images with the light source off.

19. The method of claim 17, wherein the step of measuring a skin sample via transcutaneous spatially resolved diffuse reflectance comprises measuring the skin sample using a measurement sequence comprising obtaining a plurality reflectance images with the light source on, and obtaining a plurality of background images with the light source off.

20. The method of claim 17, wherein the step of calculating a bilirubin concentration withing the skin sample comprises:
converting a plurality of raw images to a plurality of digital negative images;
demosaicing the plurality of digital negative images;
identifying and removing outlier images from the demosaiced digital negative images;
averaging the remaining demosaiced digital negative images to produce a skin reflectance image, a skin background image, a calibration reflectance image, and a calibration background image;
subtracting the background images from corresponding reflectance images;
normalizing the background subtracted skin reflectance image via the background subtracted calibration reflectance image to produce an RGB calibrated reflectance image; and
applying a generalized linear model to obtain a bilirubin concentration.

21. A non-transitory computer readable medium comprising instructions which, when executed by a computer, causes the computer to perform steps comprising:
calibrating the system of claim 1;
measuring a skin sample via transcutaneous spatially resolved diffuse reflectance;
calculating a bilirubin concentration withing the skin sample; and
displaying the bilirubin concentration.
